(19) United States
(12) Patent Application Publication
(10) Pub. No.: US 2025/0090440 A1
(43) Pub. Date: Mar. 20, 2025

(54) NOVEL CYCLIC ACETAL COMPOUNDS WITH ALDEHYDE OR KETONE FUNCTIONALITIES AND USES THEREOF

(71) Applicant: Structure Holdings, LLC, Grosse Pointe Farms, MI (US)

(72) Inventors: Sean William Raspet, San Francisco, CA (US); Shengping Zheng, Scansdale, NY (US); Sihan Li, Brooklyn, NY (US)

(73) Assignee: Structure Holdings, LLC, Grosse Pointe Farms, MI (US)

(21) Appl. No.: 18/938,387

(22) Filed: Nov. 6, 2024

Related U.S. Application Data
(63) Continuation-in-part of application No. 16/895,442, filed on Jun. 8, 2020.
(60) Provisional application No. 62/858,999, filed on Jun. 8, 2019.

(51) Int. Cl. A61K 8/49; A61Q 13/00; C07D 317/26; C07D 317/64; C07D 317/70; C07D 317/72; C07D 319/06; C07D 319/08; C07D 321/08; C07D 407/06; C07F 7/18

(52) U.S. Cl. CPC A61K 8/498; A61K 8/4973; A61Q 13/00; C07D 317/26; C07D 317/64; C07D 317/70; C07D 317/72; C07D 319/06; C07D 319/08; C07D 321/08; C07D 407/06; C07F 7/1804

(57) ABSTRACT

Novel molecules and novel fragrance molecules are disclosed, with the series of related molecules all containing a cyclic acetal or dioxepane sub-structure in combination with a carbonyl sub-structure and a cyclic structure which may be a phenyl or non-phenyl substituent. The molecules may contain further substitutions or modifications. The molecules compose or modify the sensory qualities of a fragrance, flavor, or a scented or flavored product. Further information is given on the predicted olfactory receptor activity of the invention and a method for composing novel molecular structures with regard to particular receptors of interest in order to improve the pleasantness, multidimensionality, and utility of olfactory molecules.

### CROSS-REFERENCE TO RELATED APPLICATIONS

This application claims priority under 35 USC 120 as a continuation in part of U.S. application Ser. No. 16/895,442 filed Jun. 8, 2020, which in turn claims priority under 35 USC 119(e) to U.S. Provisional Application No. 62/858,999 filed Jun. 8, 2019, the contents of both of which are incorporated by reference in their entireties.

### FIELD OF THE INVENTION

The present disclosure provides for novel compounds, compositions that include those compounds and methods of making and using those compounds. The compounds have a structure that provides the compounds with both a novel flavor and fragrance. The molecules, all contain a cyclic acetal sub-structure in combination with a carbonyl substructure and also contain another ring structure.

The present disclosure provides for novel fragrance molecules, namely a range of related fragrance molecules containing a cyclic acetal sub-structure in combination with a carbonyl sub structure.

### BACKGROUND OF THE INVENTION

Fragrances such as perfumes have been used by Homo sapiens since at least as early as about 3500 BCE, which historians believe were first utilized in Mesopotamia. Egyptians, Indians, and Cyprians were also early users of perfumes. Perfumes, flavoring, and scented products have been important through the ages and are in continuing and increasing use today. The fragrance, flavor, and scented consumer products industries, continue to seek and develop novel fragrance ingredients in order to impart new and interesting fragrance qualities to products. These novel fragrance ingredients, in particular, novel molecular compounds, allow for the differentiation of products from other products creating both new and memorable sensory experiences for consumers.

The sensory space of possible fragrance is vast and relatively unexplored. While the entirety of all possible colors in human vision are the result of the activity of three types of color receptor in the human eye, the space of scent is exponentially vaster, with an estimated ˜400 types of functioning human olfactory receptors. There are yet many undiscovered “colors” in the fragrance space.

An effective way to explore the space of fragrance is by producing novel molecular structures with novel scent properties. These allow for the production of new and useful scent-related products. Several examples of novel fragrance molecules and a method for their manufacture are disclosed herein.

In the fragrance, flavor and scented consumer products industries, there is an ongoing need for novel fragrance ingredients in order to impart new and interesting fragrance qualities to products. These novel fragrance ingredients, in particular, novel molecular compounds, can allow products to differentiate from competing products and to create, new and memorable sensory experiences for consumers. In this document, it is understood that the term fragrance also includes the related applications of flavor and other products related to olfactory and gustatory experience.

### SUMMARY OF THE INVENTION

The present invention relates to the discovery of a molecular structure template or rule that corresponds to a range of powerful new floral odorant molecules. In an embodiment, these compounds contain a 1) phenyl group that is present in a molecular structure that is then bound to 2) a cyclic acetal structure (for example: a dioxane or dioxolane) or dioxepane structure, either directly or via a chain or one or more carbon atoms, and 3) a carbonyl structure is present on or in close proximity to the cyclic acetal's ring structure. These molecules tend to have a powerful, diffusive and highly desirable floral, sweet character that is useful in fragrance and related applications.

In an embodiment, several compounds of the formula:
R—(R¹)ₙ—R²—R³ are disclosed wherein:
R=is or contains a phenyl group
R1=is or contains a methyl, ethyl, propyl, or butyl group, including branched groups or groups with additional methyl, ethyl or propyl sub-structures
R2=is or contains a cyclic acetal group or dioxepane
R3=is or contains a carbonyl group, including a ketone, methanal, ethanal, propanal, or acetyl group

And wherein the compound is novel, having not been described in prior art relating to fragrance applications.

Alternatively, and/or additionally, the present invention relates to compounds, compositions and methods using the compounds of the present invention. In an embodiment, the compounds are compounds of the formula I:

R—(R¹)ₙ—R²—R³ formula I
wherein
R comprises either a phenyl group with additional substitutions as described herein, or a non-phenyl cyclic structure with additional substitutions or modifications as described herein
R¹ is a linker moiety selected from the group consisting of a methylene, —O—CH2-, an ethylene, a propylene, and a butylene group that is optionally substituted with a methyl, a dimethyl, an ethyl, or a propyl group, and that optionally may contain a C═C double bond.
n is 0 or 1, including a linkage via a directly shared carbon such that R and R² create a polycyclic spiro structure or substructure.

R² comprises a dioxolane, dioxane or dioxepane group
R³ comprises a carbonyl moiety that is part of a C₀₋₃ ketone (including a ketone as a component of a lactone when combined with an oxo ether in R²) or a C₁₋₃ aldehyde functionality

As exemplified by the compounds disclosed herein, the present invention relates to a molecular structure template or rule that corresponds to a range of powerful and pleasant new odorant molecules. These compounds contain 1) a phenyl group with further substitutions or a non-phenyl ring structure with further substitutions or modifications present in a molecular structure that is then bound to 2) a cyclic acetal structure (for example: a dioxane or dioxolane) or dioxepane structure, either directly or via a chain or one or more carbon atoms, and 3) a carbonyl structure that is present on or in proximity to the cyclic acetal's or dioxepane's ring structure. These molecules tend to have a powerful, diffusive, highly desirable, complex, rich, sweet and full character that is useful in fragrance and related applications.

### BRIEF DESCRIPTION OF THE SEVERAL VIEWS OF THE DRAWINGS

FIG. 1 shows the molecule 2-{2-[3-(propan-2-yl)phenyl]propyl}-1,3-dioxan-5-one docked in the VN1R1 receptor (human vomeronasal 1 receptor 1) using predictive docking and protein folding software algorithms.

FIG. 2 is a close up view of 2-{2-[3-(propan-2-yl)phenyl]propyl}-1,3-dioxan-5-one (Example 1) docked in the VN1R1 receptor.

FIG. 3 is a hydrogen bond shown between the ketone group of 2-{2-[3-(propan-2-yl)phenyl]propyl}-1,3-dioxan-5-one (Example 1) and the —OH group of the Tyrosine residue #268 on the protein VN1R1.

FIG. 4 is a hydrogen bond shown between the ether group of 2-{2-[3-(propan-2-yl)phenyl]propyl}-1,3-dioxan-5-one (Example 1) and the OH group of the Tyrosine residue #224 on the protein VN1R1.

FIG. 5 is a cation-pi interaction shown between the benzene ring of 2-{2-[3-(propan-2-yl)phenyl]propyl}-1,3-dioxan-5-one (Example 1) and the guanidine group of the arginine residue #129 on the protein VN1R1.

FIG. 6 are hydrophobic interactions shown between the isopropyl tail and benzene ring of 2-{2-[3-(propan-2-yl)phenyl]propyl}-1,3-dioxan-5-one (Example 1) with various residues of VN1R1.

### DETAILED DESCRIPTION OF THE INVENTION

In this document, it is understood that the term fragrance also includes the related applications of flavor and other products related to olfactory and gustatory experience. Furthermore, applications related to therapeutics, especially those that target ectopically expressed olfactory receptors, and other applications for novel compounds contained herein are all understood to be within the scope of the invention disclosed herein.

In an embodiment, the present invention relates to the compounds of formula I:

R—(R¹)ₙ—R²-R³ formula I
wherein
R comprises either [1]:
   a phenyl group that also includes any of the following substitutions alone or in combination:
   a. One or more methyl groups
   b. A one to six carbon chain that may be linear, or branched, or including dimethyl or trimethyl moieties, and optionally including one alcohol group or one ketone group
   c. A one to six carbon chain that may be linear or branched, containing one or more double bonds, each in either cis- or trans-configuration, and optionally including one alcohol group or one ketone group
   d. An ether bonded directly to R on one side and bonded to a one- to six-carbon chain that may be linear or branched, or including dimethyl or trimethyl moieties
   e. Trimethylsilyl
   f. Trimethylsiloxy
   g. Tri-isopropylsiloxy
   h. Tri-cyclopropylsiloxy
   i. One or more halogens selected from the group consisting of fluorine, chlorine, bromine, and iodine
   j. An alcohol
   k. A phenyl bonded to two adjacent carbons on R such that R is converted to a naphthyl moiety. Said phenyl may optionally include a methyl, alcohol, or methoxy substitution
   l. A non-phenyl ring structure where said ring structure is bonded to two adjacent carbons on R such that R is converted to a planar polycyclic system. Said ring structure may optionally include one or more oxygen atoms in combination with carbon atoms. Said ring structure may optionally include one or more methyl substituents and one or more unsaturated bonds. Said ring structure may include up to seven members including the two carbons on R to which is it bonded.
   m. Ester, wherein the ester functionality is either bonded directly to R or contained within a branched or unbranched carbon chain of seven or fewer carbons in total
   With the following provisos:
      1. If R contains a methoxy substitution, that substitution must occur in combination with at least one other substitution from the above list of substitutions.
      2. The phenyl group R is not bonded to R², such that R and R² together form a planar polycyclic structure.
OR R comprises [2]:
   a cyclopropyl, cyclobutyl, cyclopentyl, cyclohexyl or cycloheptyl ring. Said ring may optionally be bonded to R² in a spiro configuration. Said ring may optionally include any of the following substitutions or modifications, either alone or in combination:
   a. One or more C═C double bonds within said ring, provided that said ring itself is not a phenyl group
   b. One or more methyl groups attached to said ring
   c. A one to six carbon chain that may be linear, or branched, or including dimethyl or trimethyl moieties, and optionally including one alcohol group or one ketone group connected to said chain.
   d. One or more carbon bridge atoms such that R is converted to a polycyclic structure, such that said polycyclic structure does not consist of more than 17 heavy atoms
   e. A phenyl group bonded to said ring sharing two carbons such that the phenyl group creates a polycyclic planar structure in conjunction with said ring.
   f. Ester, wherein the ester functionality is either bonded directly to R or contained within a branched or unbranched carbon chain of seven or fewer carbons in total
   g. A ketone group bonded directly to R
   h. A one to seven carbon structure that contains an ether group. Said ether group may be directly bonded to R or may be located between two other carbons within said structure. Said structure may be linear or branched, or including dimethyl or trimethyl moieties.
   With the following proviso:
      1. If the ring structure is a cyclohexane, R² and R³ do not together comprise a dioxolane structure with a directly attached ketone wherein the structure of R² and R³ is directly bonded to R in a manner that creates a spiro configuration
R¹ is a linker moiety selected from the group consisting of a methylene, —O—CH2-, an ethylene, a propylene, and a butylene group that is optionally substituted with a methyl, a dimethyl, an ethyl, or a propyl group, and that optionally may contain a C═C double bond.
n is 0 or 1, including a linkage via a directly shared carbon such that R and R² create a polycyclic spiro structure or substructure.
R² comprises a dioxolane, dioxane or dioxepane group
R³ comprises a carbonyl moiety that is part of a C₀₋₃ ketone (including a ketone as a component of a lactone when combined with an ether in R²) or a C₁₋₃ aldehyde functionality
   In an embodiment, the present invention relates to the compounds of formula II.

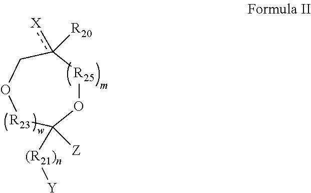

wherein X is selected from the group consisting of O and —(CH₂)ᵥCHO, wherein v is 0, 1, or 2; and
when X is O, R₂₀ is not present;
R₂₀ is selected from the group consisting of H and C₁₋₃alkyl, or R₂₀ is not present; m is 0, 1, or 2, and
n is 0, 1, or 2;
R₂₁ is selected from the group consisting of —CH₂—, —R₂₂—, —OR₂₂—, —CH₂R₂₂—, and —R₂₂CH₂— wherein
R₂₂ is C₁₋₆alkyl, or C₁₋₃alkylene optionally substituted with a C₁₋₃alkyl;
Y is selected from the group consisting of phenyl, C₃₋₆cycloalkyl, C₃₋₆cycloalkenyl, cyclodienalkyl, adamantyl, naphthalenyl, dihydronaphthalenyl, tetrahydronaphthalenyl, benzodioxolyl, bicyclo[2.2.2]alkyl, bicyclo[3.1.1]alkyl, bicyclo[2.2.2]alkenyl, bicyclo[3.1.1]alkenyl; or when n=0 and Z is not present, Y is a spiro-linked C₃₋₆cycloalkyl, C₃₋₆cycloalkenyl, or cyclodienalkyl; and Z is hydrogen, C₁₋₃alkyl, or not present; wherein Y is optionally substituted with one or more substituents independently selected from the group consisting of hydrogen, hydroxyl, —C(O)OC₁₋₃alkyl, —C₁₋₆alkyl, —C₁₋₆alkenyl, —O—C₁₋₆alkyl, halogen, or —O—Si(CH₃)₃ wherein the —C₁₋₆alkyl and —C₁₋₆ alkenyl as optional substituents off of Y are optionally substituted with hydroxyl, and R₂₃ is (—CH₂—)_{w} wherein w is 0 or 1, or R₂₁, R₂₃, and Y together form a ring structure, R₂₅ is —CH₂— or —CHR₂₆— where R₂₆ is C₁₋₃alkyl.

In an embodiment, R₂₁ and R₂₃ together with the atoms to which they are attached form a five membered ring structure which is optionally fused to a phenyl group.

In an embodiment, halogen is bromo. In a variation, m is 0. In a variation, m is 1. In a variation, the one or more optional substituents on Y is one of more of bromo, methyl, ethyl, propyl, isopropyl, n-butyl, sec-butyl, isobutyl, or t-butyl.

Several examples of compounds of which the invention consists as well as potential chemical reactions to synthesize a selection of some of the compounds of which the invention consists are included herein.

In the case of the chemical synthesis reactions described below it is understood that the invention may be synthesized via other reactions. The reactions given herein are provided as examples and should not limit the scope of the invention.

The embodiments detailed below are provided as examples and should not limit the scope of the invention.

### EXAMPLES

### Example 1

The following example is intended to be representative of an embodiment of the present invention. A molecule of the formula:

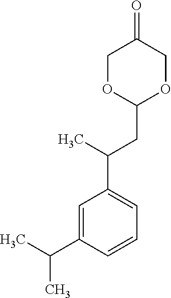 [SMILES: CC(C)c1cccc(C(C)CC2OCC(=O)CO2)c1]

### 2-{2-[3-(propan-2-yl)phenyl]propyl}-1,3-dioxan-5-one

In one embodiment, the compound above can be produced via oxidation of 2-{2-[3-(propan-2-yl)phenyl]propyl}-1,3-dioxan-5-ol.

2-{2-[3-(propan-2-yl)phenyl]propyl}-1,3-dioxan-5-ol itself can be produced via a cyclic acetal formation between glycerol and the aldehyde 3-[3-(propan-2-yl)phenyl]butanal. In this document it is understood that the term acetal is used as a general term to designate both acetals and ketals.

One of the products from the above reaction is the cyclic acetal, alcohol-containing compound: 2-{2-[3-(propan-2-yl)phenyl]propyl}-1,3-dioxan-5-ol. This compound is then oxidized to form the embodiment 2-{2-[3-(propan-2-yl)phenyl]propyl}-1,3-dioxan-5-one.

The reaction proceeds as in the scheme below.

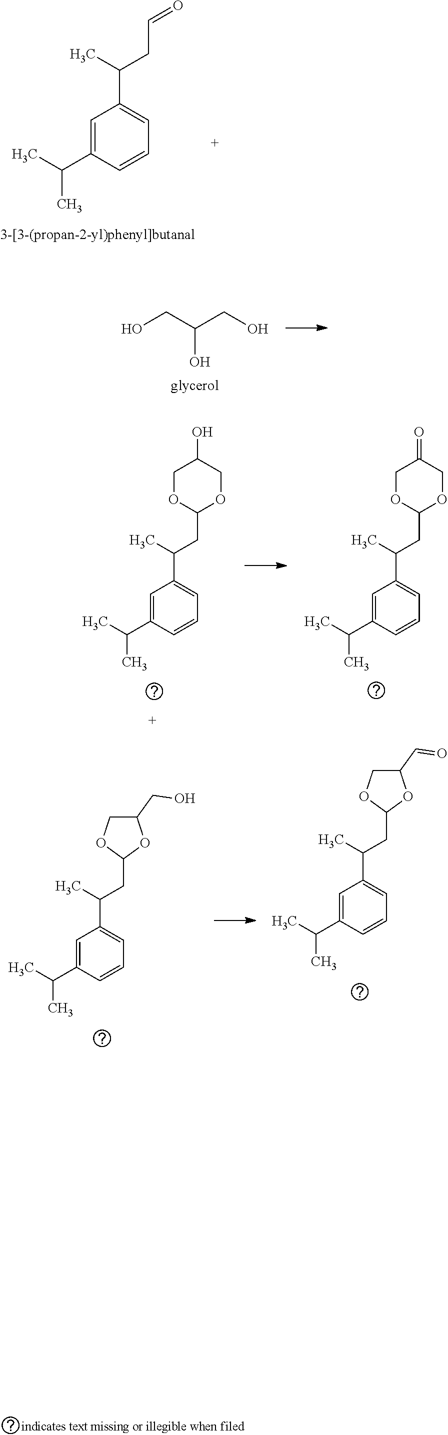 [SMILES: CC(C)c1cccc(C(C)CC=O)c1 ; OCC(O)CO ; CC(C)c1cccc(C(C)CC2OCC(O)CO2)c1 ; CC(C)c1cccc(C(C)CC2OCC(=O)CO2)c1 ; CC(C)c1cccc(C(C)CC2OCC(CO)O2)c1 ; CC(C)c1cccc(C(C)CC2OCC(C=O)O2)c1]

2-{2-[3-(propan-2-yl)phenyl]propyl}-1,3-dioxan-5-one has a very pleasant, complex odor profile that has been described as aquatic, green, and floral but with a floral-sweet jasmine-like lactonic aspect. Others have described these molecules as being reminiscent of the rich, green and yet ozonic aspect of matcha tea, also being reminiscent of matcha cakes with their more lactonic, sweet, baked character.

Compared to the starting material 3-[3-(propan-2-yl)phenyl]butanal it has a surprisingly complex and naturalistic character. While the starting material has been described as having an aquatic aspect, it has also been commonly described as rubbery, plastic, and synthetic seeming. 2-{2-[3-(propan-2-yl)phenyl]propyl}-1,3-dioxan-5-one in comparison to the starting material has been reviewed as having much more depth, strength and softness whereas the starting material 3-[3-(propan-2-yl)phenyl]butanal is described as ‘thin,’ ‘one-note’ and ‘hollow’.

The compound 2-{2-[3-(propan-2-yl)phenyl]propyl}-1,3-dioxan-5-one is very powerful and diffusive which can offer the advantage of a lower use percentage and thus a lower cost-in-use in formulations. In addition to its use in formulations it can potentially be used as a stand-alone note or single-molecule fragrance because of its additional naturalism and complexity.

In other compositions it may provide a base for or aspects of spring air, forest freshness and other fresh, floral-sweet aquatic notes.

Without being bound by theory, a reason for 2-{2-[3-(propan-2-yl)phenyl]propyl}-1,3-dioxan-5-one's complex, pleasant and multifaceted character may be its strong affinity for one or more human vomeronasal receptors, in particular the VN1R1 receptor. The specifics of this binding are described elsewhere in this application, as is the novel use of targeting a specific olfactory receptor and in particular a vomeronasal receptor in the design and validation process of an odorant molecule. It should be understood that although the binding is shown with one particular compound, the present invention should not be construed to be limited to only that compound. Because of the similarities in structure of the recited compounds, it should be understood that any of the plurality of compounds disclosed herein are contemplated as being able to bind the receptors as disclosed herein.

In one embodiment 3-[3-(propan-2-yl)phenyl]butanal (10 mmol, 1 equiv) is added to freshly distilled THE (20 ml), to which glycerol (10 mmol, 1 equiv) is then added. The mixture is then stirred at room temperature for 5 min following which, FeCl3 is added. The solvent is then heated under reflux conditions for 12 hours.

When finished, the solution is a brown color and extracted with CH2Cl2. A column can be used to purify the crude product which yields the step 1 alcohol 2-{2-[3-(propan-2-yl)phenyl]propyl}-1,3-dioxan-5-ol. The freshly made step 1 alcohol product is then used to produce the 2-{2-[3-(propan-2-yl)phenyl]propyl}-1,3-dioxan-5-one ketone using the following scheme:

In a vessel in an ice bath, add the newly made alcohol (1 equiv), NH4tButylSO4 (5%), and TEMPO (1%) in CH2Cl2. The mixture solution is then stirred for 10 minutes and then NaOCl5H2O is slowly added in 5 portions over 20 minutes.

The solution is then removed to room temperature and stirred for 2 hours. When the reaction is finished, water is added, and it is extracted with CH₂Cl₂. The crude product can be purified with a column to isolate 2-{2-[3-(propan-2-yl)phenyl]propyl}-1,3-dioxan-5-one

### NMR Spectra for Example 1:

¹H NMR (400 MHz): δ 1.03-1.24 (9H, 1.09 (d, J=6.9 Hz), 1.18 (d, J=6.9 Hz)), 1.79 (2H, dd, J=10.1, 5.8 Hz), 2.89 (1H, tq, J=10.1, 6.9 Hz), 3.02 (1H, sept, J=6.9 Hz), 4.57 (4H, d, J=16.0 Hz), 5.09 (1H, t, J=5.8 Hz), 6.94-7.08 (2H, 7.00 (ddd, J=7.9, 2.6, 2.4 Hz), 7.01 (ddd, J=7.9, 2.6, 2.3 Hz)), 7.14-7.37 (2H, 7.20 (td, J=7.9, 0.5 Hz), 7.31 (ddd, J=2.4, 2.3, 0.5 Hz)).

### Example 2

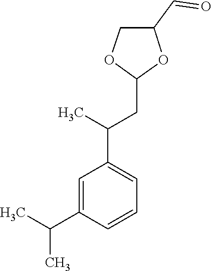 [SMILES: CC(C)c1cccc(C(C)CC2OCC(C=O)O2)c1]

### 2-{2-[3-(propan-2-yl)phenyl]propyl}-1,3-dioxolane-4-carbaldehyde

The above embodiment can be prepared as a side product of the synthesis scheme (shown above) for 2-{2-[3-(propan-2-yl)phenyl]propyl}-1,3-dioxan-5-one.

It may serve as a useful product in combination 2-{2-[3-(propan-2-yl)phenyl]propyl}-1,3-dioxan-5-one, which would allow for streamlining the synthesis steps involved in isolating each compound individually. With a similar yet distinct, aquatic lactonic character, it may serve as a useful, desirable product in its own right.

### NMR Spectra for Example 2:

¹H NMR (400 MHz): δ 1.03-1.24 (9H, 1.09 (d, J=6.9 Hz), 1.18 (d, J=6.9 Hz)), 1.78 (2H, dd, J=10.1, 6.2 Hz), 2.89 (1H, tq, J=10.1, 6.9 Hz), 3.02 (1H, sept, J=6.9 Hz), 3.93 (2H, dd, J=14.7, 7.4 Hz), 4.81 (1H, ddd, J=9.2, 5.7, 4.6 Hz), 5.03 (1H, t, J=6.2 Hz), 6.94-7.08 (2H, 7.00 (ddd, J=7.9, 2.6, 2.4 Hz), 7.01 (ddd, J=7.9, 2.6, 2.3 Hz)), 7.14-7.37 (2H, 7.20 (td, J=7.9, 0.5 Hz), 7.31 (ddd, J=2.4, 2.3, 0.5 Hz)), 9.63 (1H, d, J=4.6 Hz).

### Example 3

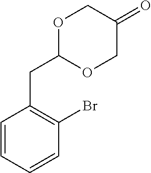 [SMILES: O=C1COC(Cc2ccccc2Br)OC1]

### 2-[(2-bromophenyl)methyl]-1,3-dioxan-5-one

In another embodiment R consists of a phenyl group that is substituted with a bromine atom at the ortho position to R¹. In this embodiment, R² and R³ together form a 1,3-dioxan-5-one structure, wherein the R¹ linker moiety is bonded to the 2 position on the 1,3-dioxan-5-one ring.

This embodiment has been described as having a strong, diffusive and highly realistic watery aspect. It has been described as having a much more faithful and convincing watery facet than other well-known fragrance materials such as 7-Methyl-2,4-dihydro-3H-1,5-benzodioxepin-3-one (“Calone®”) and 4-(4,8-Dimethyl-3,7-nonadienyl)-pyridine (“Maritima®”)

This embodiment can be produced via the following synthesis scheme with the method used to synthesize 2-{2-[3-(propan-2-yl)phenyl]propyl}-1,3-dioxan-5-one applied to the commercially available starting materials 2-(2-bromophenyl)acetaldehyde and glycerol.

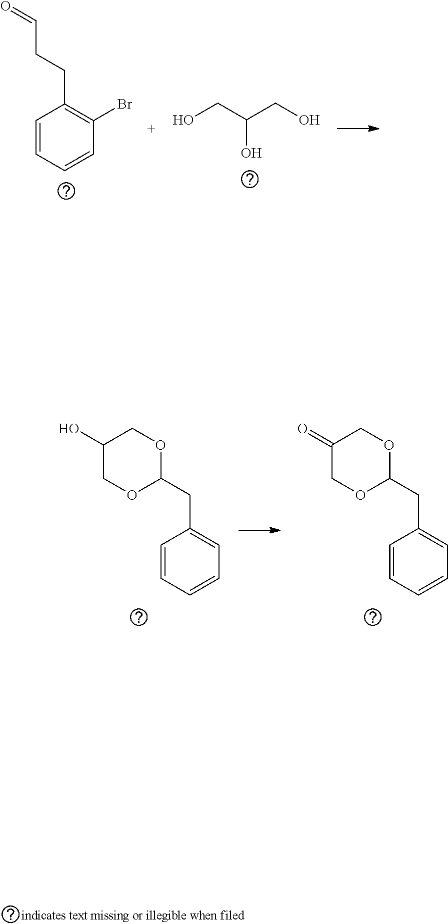 [SMILES: O=CCCc1ccccc1Br ; OCC(O)CO ; OC1COC(Cc2ccccc2)OC1 ; O=C1COC(Cc2ccccc2)OC1]

### Example 4

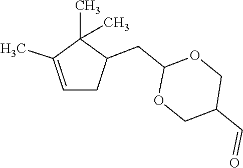 [SMILES: CC1=CCC(CC2OCC(C=O)CO2)C1(C)C]

### 2-[(2,2,3-trimethylcyclopent-3-en-1-yl)methyl]-1,3-dioxane-5-carbaldehyde

Another embodiment of the invention consists of an R group that is a trimethyl cyclopentene. This R group is then bonded to a R² and R³ 1,3-dioxane-5-carbaldehyde structure at via a one carbon R¹ that connects to the 2 position of the 1,3-dioxane-5-carbaldehyde.

In comparison to the starting material, 2-(2,2,3-trimethylcyclopent-3-en-1-yl)acetaldehyde (“alpha-campholenic aldehyde”) which has been described as herbal and green with aspects of perilla leaf, the embodiment 2-[(2,2,3-trimethylcyclopent-3-en-1-yl)methyl]-1,3-dioxane-5-carbaldehyde adds a great deal of complexity and richness to the base profile. At the same time, it provides a green ‘tea tree’ facet that is not present in the original aldehyde, along with a very naturalistic woody, sap or resin aspect slightly reminiscent of incense. An additional facet that it contains has been described as the velvety aspect of patchouli and an old wood, dry airiness.

This embodiment can be produced via the following synthesis scheme with the method used to synthesize 2-{2-[3-(propan-2-yl)phenyl]propyl}-1,3-dioxan-5-one applied to the commercially available starting materials 2-(2,2,3-trimethylcyclopent-3-en-1-yl)acetaldehyde and 2-(hydroxymethyl)propane-1,3-diol.

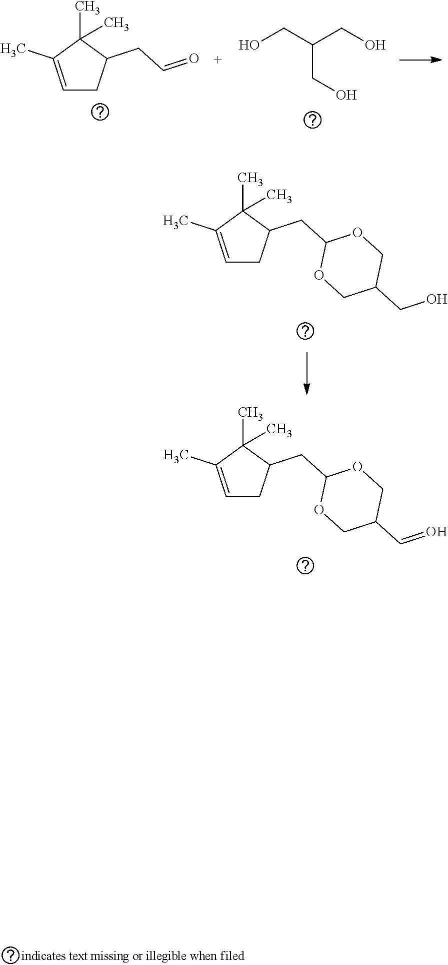 [SMILES: CC1=CCC(CC=O)C1(C)C ; OCC(CO)CO ; CC1=CCC(CC2OCC(CO)CO2)C1(C)C ; CC1=CCC(CC2OCC(C=O)CO2)C1(C)C]

### Example 5

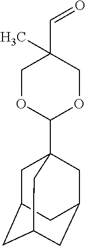 [SMILES: CC1(C=O)COC(C23CC4CC(CC(C4)C2)C3)OC1]

### 2-(adamantan-1-yl)-5-methyl-1,3-dioxane-5-carbaldehyde

Another embodiment of the invention—2-(adamantan-1-yl)-5-methyl-1,3-dioxane-5-carbaldehyde—consists of an R group that is an adamantane group. This adamantane group is then bonded directly to an R² and R³ 5-methyl-1,3-dioxane-5-carbaldehyde structure at via a zero-carbon R¹ that connects to the 2 position of the 5-methyl-1,3-dioxane-5-carbaldehyde.

This embodiment can be produced via the following synthesis scheme with the method used to synthesize 2-{2-[3-(propan-2-yl)phenyl]propyl}-1,3-dioxan-5-one applied to the commercially available starting materials adamantane-1-carbaldehyde and 2-(hydroxymethyl)-2-methylpropane-1,3-diol.

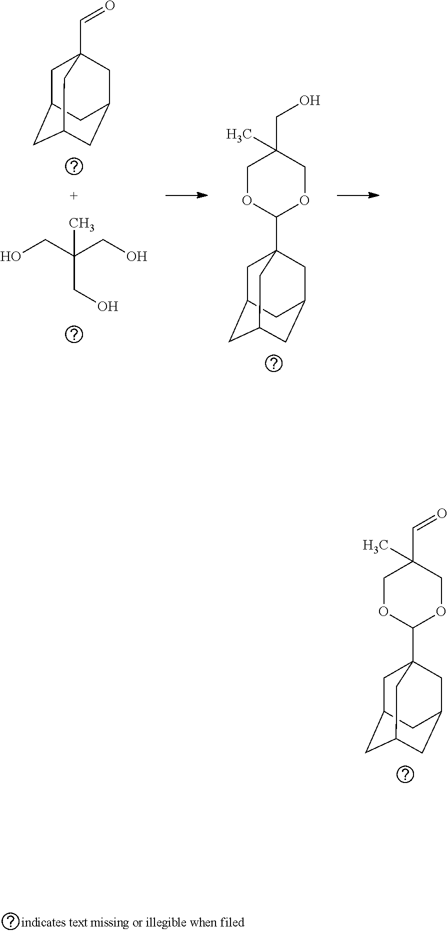

### Generic Synthesis Example

One method of synthesizing many compounds of the formula disclosed herein is with the following scheme. It takes as a starting material 1) a molecule with an aldehyde or ketone carbonyl functional group along with the R—(R¹)ₙ groups detailed above. Here the carbonyl is at the position of what will become the R² group.

This carbonyl group is reacted with a molecule containing 3 alcohol groups to create a cyclic acetal or dioxepane with a remaining alcohol group attached to it. Examples of these include but are not limited to glycerol, 2-(hydroxymethyl)propane-1,3-diol, 2-(hydroxymethyl)-2-methylpropane-1,3-diol, and butane-1,2,4-triol.

Following this, the alcohol product with the cyclic acetal structure is further oxidized to create a cyclic acetal or dioxepane structure with an attached carbonyl.

R—(R¹)ₙ

In the scheme below a generic scheme is shown for an R—(R¹)ₙ-aldehyde starting material reacted with each of the triols glycerol, 2-(hydroxymethyl)propane-1,3-diol, 2-(hydroxymethyl)-2-methylpropane-1,3-diol, and butane-1,2,4-triol to show the intermediate products of the cyclic acetal or dioxepane structure with an additional alcohol group. Following this, the scheme shows the oxidation of the intermediate alcohol to a cyclic acetal or dioxepane structure with an attached carbonyl group. In this case the carbonyl group is an aldehyde in each example in the scheme.

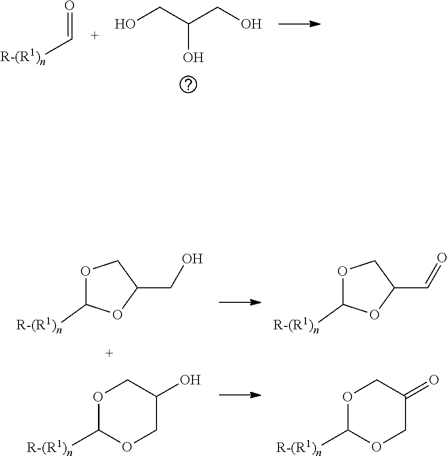

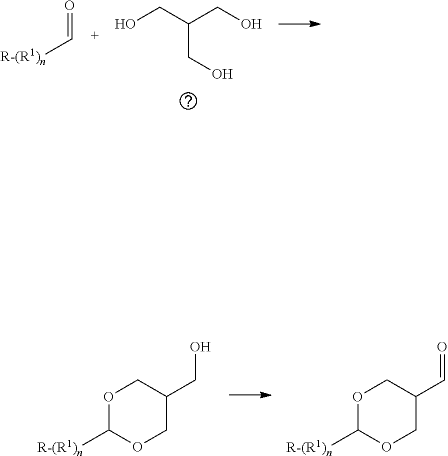

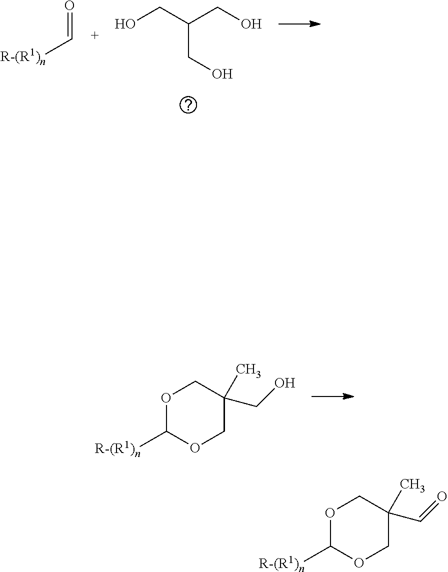

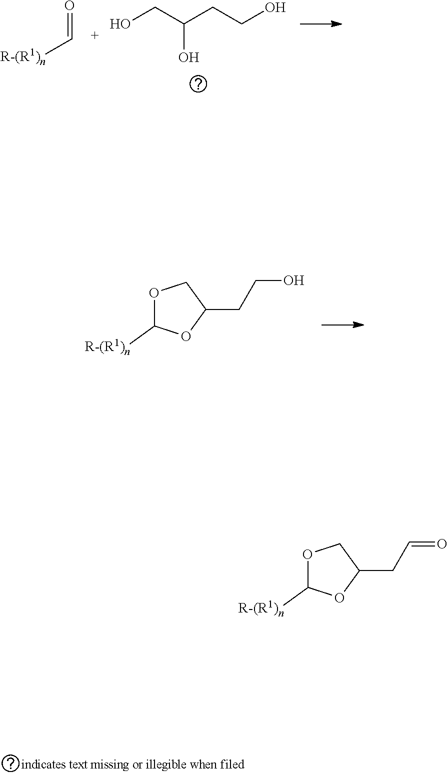

In its broadest sense, the compounds of the present invention can be made by reacting an aldehyde with a glycerol compound or some other alkyl triol component. For example, the reaction of benzaldehyde with glycerol will generate a phenyl group attached to a dioxane or dioxolane.

### Generic Synthesis for Dioxepane Embodiments

In the case of embodiments of the invention that contain dioxepane sub-structures, one method of synthesis proceeds with a diol that is connected to the R—(R¹)ₙ sub-structure, rather than an aldehyde or ketone. In this scheme, the diol is first reacted with chlorotri(methyl) silane to produce a trimethylsiloxy intermediate. This intermediate is then reacted with a dialdehyde that also contains an alcohol group. The reaction forms a closed ring such as a diopxepan-ol or dioxepane-yl-methanol, which then with oxidation generates a ketone or aldehyde 7-ring substructure embodiment of the invention. This method is shown below.

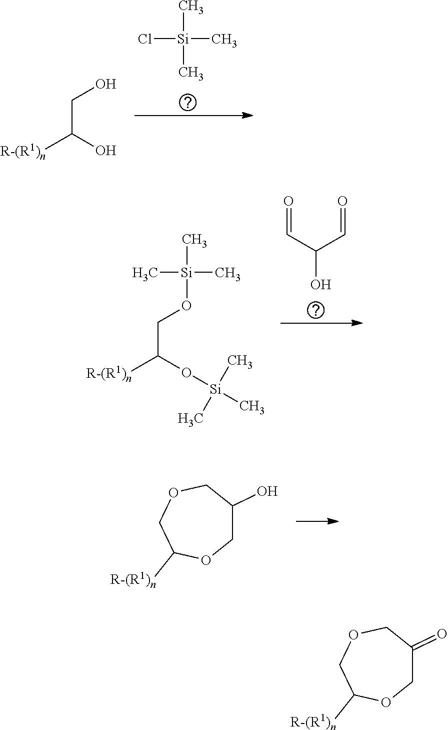

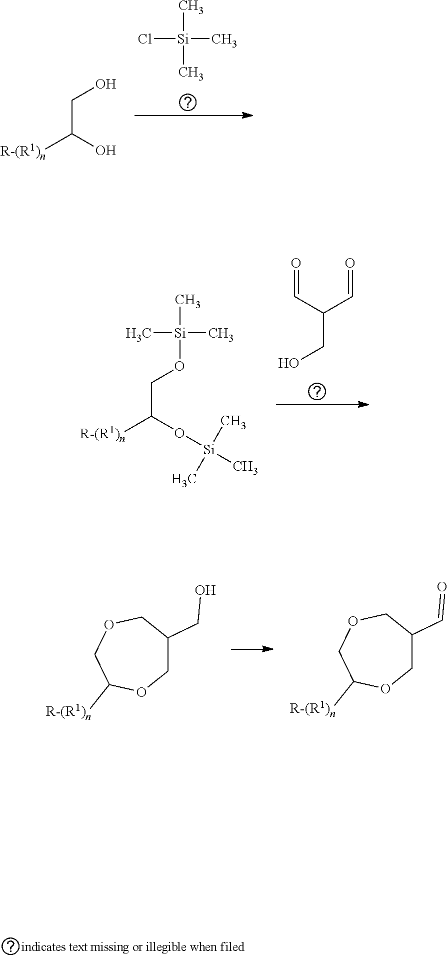

Alternatively and or additionally, the methodology follows a synthetic scheme following the Savela et al., Synthesis, 2015, 47, 1749-1760 methodology. The Savela scheme is as follows.

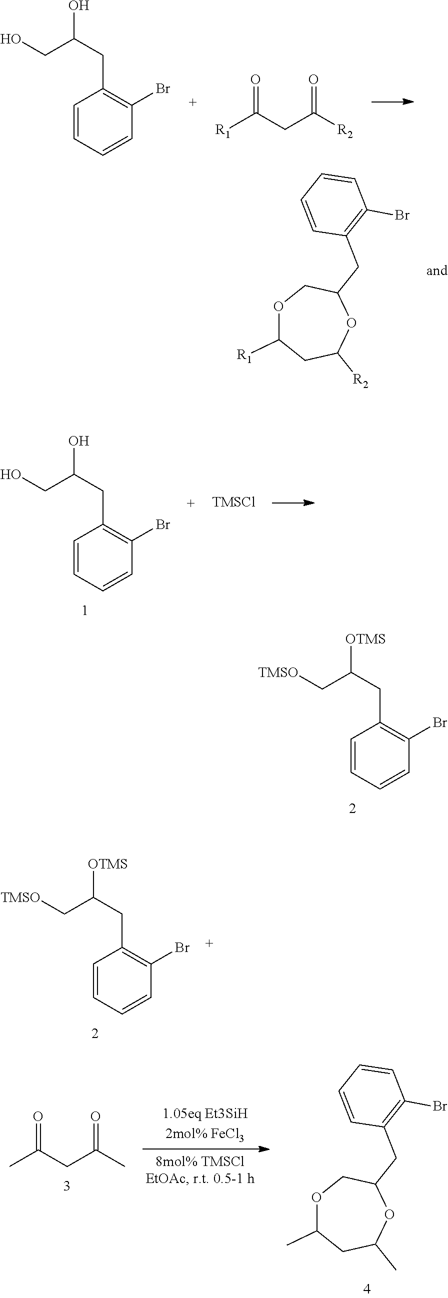

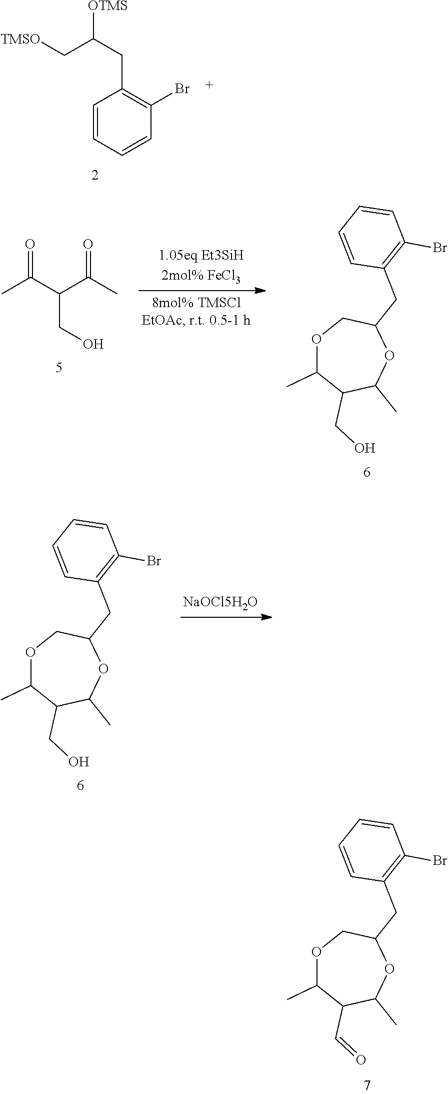

In the above scheme, diol 1 can be protected by TMSCl to generate product 2. Based on Savela's method, mixing product 2 with dione 3 is able to generate new 7-membered ring products 4. Alternatively and/or additionally, if dione 5 is chosen as a starting reagent, alcohol product 6 can be generated. This new alcohol product 6 can undergo oxidation using, for example, NaOCl·5H2O to generate a final product 7.

### Example Applications

While the preceding section has focused on the flavor or fragrance properties of the invention, it should be noted that recent discoveries in transcriptomics have demonstrated that olfactory receptors on which the human sense of smell is based, are also expressed throughout the human body in numerous other tissues outside of the olfactory epithelium. These ectopically expressed or ectopic olfactory receptors thus can serve as potential drug targets for the development of new therapeutics.

Another embodiment of this invention thus consists of the application of any of the molecular structures to which it pertains as therapeutic molecules targeting these ectopic olfactory receptors. Furthermore, olfactory sensory neurons with their corresponding receptors may themselves constitute a target for therapies and embodiments targeting these are also included within the scope of this invention.

Targets other than ectopic olfactory receptors, such as other receptors or enzymes constitute an additional category of the invention.

Relatedly, molecules that interact with the olfactory system of humans also have a likelihood of interacting with the chemical senses and overall biology of other organisms. As such another category of embodiments of this invention is the application of the invention towards other uses related to the chemical senses of organisms or their overall biology. These include antibiotics, pest repellants or attractants, plant growth stimulators, pheromones, veterinary or plant medicines, and other uses.

For novel molecular structures that are embodiments of this invention, other relevant uses outside of flavor, fragrance and medicine are also considered as within the scope of this invention's embodiments.

A further embodiment of this invention is as a method for improving or modifying the olfactory properties of a molecule that contains an aldehyde or ketone functional group by converting said aldehyde or ketone to a dioxolane, dioxane or dioxepane structure wherein said dioxolane, dioxane or dioxepane structure also has a carbonyl moiety that is part of a C₀₋₃ ketone or a C₁₋₃ aldehyde functionality directly bonded to it. Without being bound to theory, this modification of an aldehyde or ketone structure provides additional hydrogen bond acceptors in the modified version of the molecule which facilitate the binding mechanics of the molecule to the receptors that it activates. Additionally, this modification may allow the molecule to bind to and activate receptors that aren't significantly activated by the original version of the molecule. Without being bound to theory, this may result in a more complex and multifaceted sensory aspect of the modified molecule, both of which are desirable improvements in the context of flavor and fragrance.

Without being bound to theory, the same modification can provide additional binding affinity or other modification thereof for molecules targeting ectopic olfactory receptors and non-olfactory receptor therapeutic targets. A further embodiment of the invention is thus as a method for improving the binding affinity for therapeutic molecules that contain an aldehyde or ketone group by modifying said aldehyde or ketone group by converting said aldehyde or ketone to a dioxolane, dioxane or dioxepane structure wherein said dioxolane, dioxane or dioxepane structure also has a carbonyl moiety that is part of a C₀₋₃ ketone or a C₁₋₃ aldehyde functionality directly bonded to it.

The applications and embodiments disclosed herein are understood to not be exhaustive or limiting to the scope of the applications or embodiments of the invention.

### Olfactory Molecule Development Embodiments

As mentioned elsewhere in this application, the molecule 2-{2-[3-(propan-2-yl)phenyl]propyl}-1,3-dioxan-5-one (Example 1), has a high predicted binding affinity to the human vomeronasal 1 receptor 1 (VN1R1). Without being bound to theory, it is proposed that this high binding affinity is a significant contributing factor to 2-{2-[3-(propan-2-yl)phenyl]propyl}-1,3-dioxan-5-one's complex, multifaceted and pleasant odor profile.

With the recent discovery of functional vomeronasal receptors in humans, some research has been published theorizing the existence of functional human pheromones that activate these receptors and possible activity of other small molecules on these receptors. This research has focused on specific, easily quantifiable behavior changes or physiological responses, as might be expected with pheromone functioning in species other than humans. However, this research has not considered or proposed the possibility of an aesthetic, sensory aspect of vomeronasal receptor activation in humans, either alone or in combination with other odorants. The targeting of the activation of VN1R1 and other vomeronasal receptors is both a useful and novel technique in the development of improved, useful olfactory molecules.

Specifically, without being bound to theory, it is proposed that the activation of VN1R1 and of other vomeronasal receptors correlates with an olfactory sensory experience that is multifaceted and complex as well as pleasant. Moreover, without being bound by theory, it is postulated that the activation of VN1R1 and other vomeronasal receptors produces a modification and amplification of the effect and ‘fullness’ of a given odorant mixture of which it is part. This makes odorants that activate VN1R1 and other vomeronasal receptors particularly valuable to the flavor and fragrance industry.

This observation takes into account the fact that what makes a fragrance or flavor successful and desirable is not simply the rated pleasantness of each individual molecule of which it consists, but rather the complexity of sensory experience of the fragrance or flavor, which typically consists on some level of pleasant along with some unpleasant (in isolation) facets. This fact, which has not been accounted for in previous research, explains why people do not, for example, simply apply perfumes that consist solely of the molecule vanillin, which individually has been rated as the most pleasant-smelling molecule across cultures according to multiple studies. The pleasantness of vanillin alone does not satisfactorily function as a perfume because it lacks complexity, nuance and a multifaceted sensory profile. It potentially becomes cloying to the point of being sickening after extended exposure in the absence of other multidimensional olfactory facets.

Similarly, this fact explains why one of the most historically sought-after fragrance ingredients, that of jasmine, also contains the molecule indole, which is also a primary contributor to the odor of feces along with a bouquet of other traditionally pleasant-smelling molecules such as benzyl acetate, linalool and methyl jasmonate. These pleasant-smelling molecules found in jasmine do not by themselves create a satisfactory fragrance profile, but rather, when the overall mixture is enhanced by some molecules that serve as counterpoints to the floral dimension of the overall mixture (as in the case of indole), the complexity that emerges is what makes for a desirable and multidimensional olfactory experience. The pursuit of complexity and multidimensionality is central to fragrance desirability and the invention herein disclosed enhances the perceived complexity and multidimensionality of a fragrance or olfactory composition.

By taking together these insights along with recent research on olfactory receptors, new and useful compounds and methods have been discovered. The synthetic methodologies are also shown herein, which allow for the creation of desirable olfactory molecules, which can be assayed by their ability to target and bind vomeronasal receptors as a screening and optimization method. Thus, in an embodiment, the present invention relates to methods and to compounds as disclosed herein.

A molecule that can enhance this signal and complexity of a fragrance or flavor, such as 2-{2-[3-(propan-2-yl)phenyl]propyl}-1,3-dioxan-5-one and other embodiments of the invention disclosed herein, would function in a way that is analogous to the effect of monosodium glutamate (MSG) on human ‘umami’ taste receptors, which include primarily the TAS1R1+TAS1R3 heterodimer functioning in tandem, among other receptors. In particular VN1R1 and other vomeronasal receptors can be compared to the activity of the TAS1R1+TAS1R3 heterodimer with regard to glutamate in that it is correlated with the ‘synergism’ aspect of the sensory experience of glutamate when it is combined with other flavor and taste-active molecules. Like glutamate, it amplifies, unifies and makes more nuanced or complex the experience of a given olfactory experience.

In brief and without being bound to theory, VN1R1 and other vomeronasal receptors impart synergism to the overall olfactory mixture of which they are a part, making them valuable targets for the development and evaluation of new flavor and fragrance molecules (herein understood to also include the repurposing of or new use of existing molecules in a flavor and fragrance context where these molecules are not already known and/or utilized).

In a silico docking simulation for 2-{2-[3-(propan-2-yl)phenyl]propyl}-1,3-dioxan-5-one and other odorants, it has been found that 2-{2-[3-(propan-2-yl)phenyl]propyl}-1,3-dioxan-5-one has a predicted binding affinity of −7.237 kcal/mol (average of three repetitions) to the human VN1R1 receptor. This was higher than any other molecule tested. Methyl dihydrojasmonate is described as a pleasant, jasmine-floral smelling molecule with medium odor intensity and it is one of the more widely used molecules in perfumery. By comparison, the molecule methyl dihydrojasmonate (Hedione® and Kharismal® (Firmenich, Geneva Switzerland)) has been predicted to have a binding affinity to VN1R1 of −5.451 kcal/mol (average of three repetitions) which is a significantly less potent binding affinity than that of 2-{2-[3-(propan-2-yl)phenyl]propyl}-1,3-dioxan-5-one.

Other molecules that were tested, all of which are naturally occurring known odor molecules, ranged from −3.428 kcal/mol in the case of heptanal to −5.157 kcal/mol in the case of methyl jasmonate (a structural analog of Hedione®). The predicted affinity of these naturally occurring molecules broadly correlated with existing experimental literature and with Hedione®'s (Firmenich, Geneva Switzerland) relative range of activation of VN1R1, further substantiating the claim of 2-{2-[3-(propan-2-yl)phenyl]propyl}-1,3-dioxan-5-one's significantly improved activation of VN1R1 over Hedione® and other molecules and of this activation's role in the improved sensory profile of 2-{2-[3-(propan-2-yl)phenyl]propyl}-1,3-dioxan-5-one.

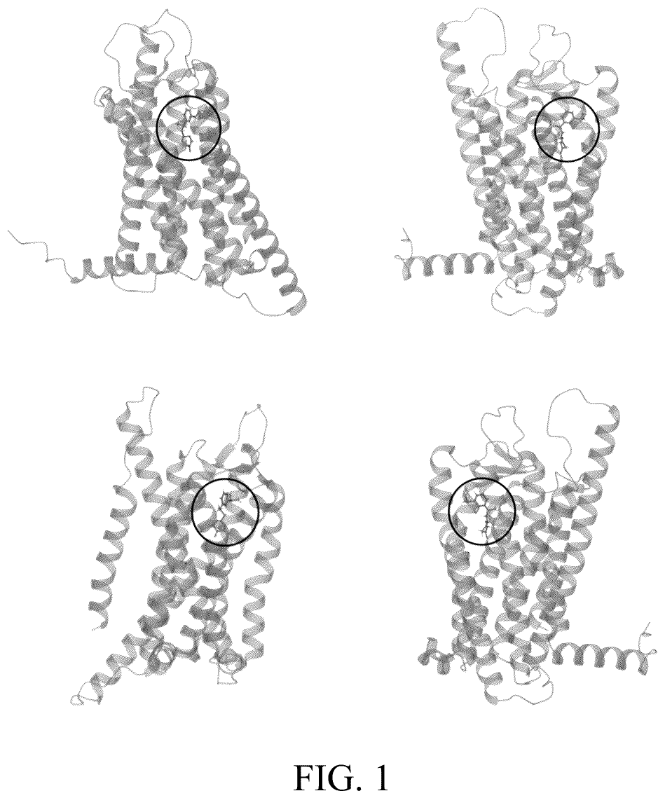

Images generated from the docking simulation of 2-{2-[3-(propan-2-yl)phenyl]propyl}-1,3-dioxan-5-one to VN1R1 are shown in FIGS. 1-6 and the additional information regarding these images is found in the “BRIEF DESCRIPTION OF THE DRAWINGS” section herein.

Without being bound to theory, this potent affinity of 2-{2-[3-(propan-2-yl)phenyl]propyl}-1,3-dioxan-5-one likely benefits from the embodiment of the invention disclosed herein wherein the overall olfactory impression and complexity of a given molecular substructure can be improved by including a substructure consisting of a dioxolane, dioxane or dioxepane structure wherein said dioxolane, dioxane or dioxepane structure also has a carbonyl moiety that is part of a C₀₋₃ ketone or a C₁₋₃ aldehyde functionality directly bonded to it.

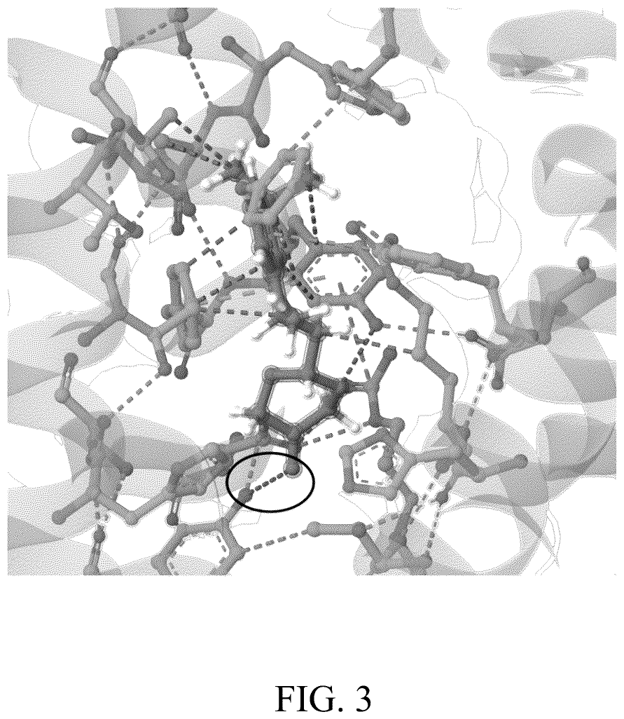

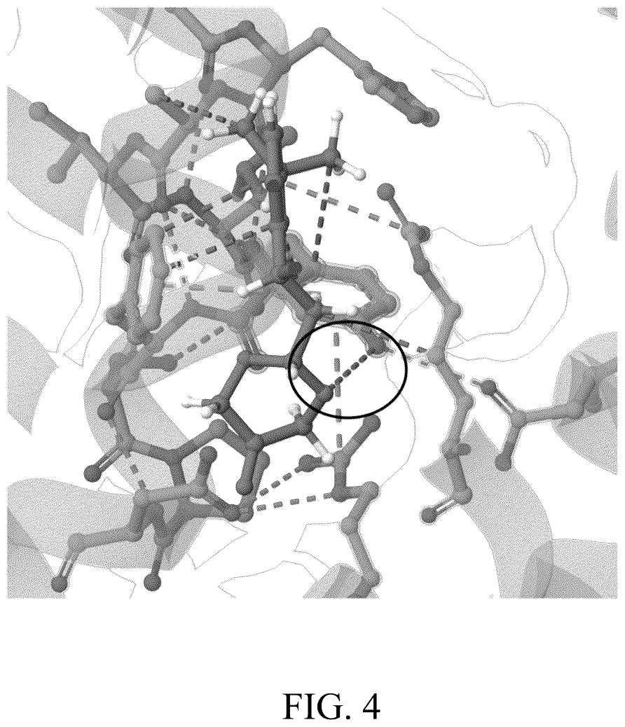

As can be observed in FIG. 3 and FIG. 4, the substructure of 2-{2-[3-(propan-2-yl)phenyl]propyl}-1,3-dioxan-5-one is able to receive two hydrogen bonds from two separate tyrosine residues within VN1R1. This additional binding potential versus a single aldehyde functional group, for example, is likely to create a stronger affinity to VN1R1 specifically and to olfactory receptors more generally in the case of 2-{2-[3-(propan-2-yl)phenyl]propyl}-1,3-dioxan-5-one as well as other embodiments of the invention disclosed herein.

In the case of this invention, the development and screening of olfactory molecules takes a novel approach by utilizing techniques from the fields of drug discovery that have not previously been associated with the development of olfactory molecules. In particular, by utilizing in silico techniques for modeling receptor conformations and ligand binding affinity to said receptors, it is possible to create novel and improved olfactory molecules. An additional embodiment of the invention disclosed herein is the novel use of molecular docking simulations for the development of olfactory molecules. These molecular docking simulations include but are not limited to protein sequence folding predictions, binding pocket identification, and ligand docking to said folded protein where the protein is an olfactory receptor (including vomeronasal receptors) and the ligand is an olfactory molecule (i.e. any molecule that exhibits an olfactory response), whether novel or existing.

Importantly, these molecular docking simulations can utilize sequence to structure protein folding predictions. Versions of this sequence to structure protein folding predictions include AlphaFold, ESMFold or ESM, OpenFold, Uni-Fold, and CollabFold among others. A key advantage of this approach and a novel aspect of this embodiment of the invention is that the majority of olfactory receptors do not have a well characterized 3D structure. Olfactory receptors all fall under the category of G protein-coupled Receptors (GPCRs) which are membrane-bound proteins. There have been significant challenges in understanding 3-dimensional structure of GPCRs in part because of their flexible structure and ability to shift conformation, along with details related to their embedding in membranes. The advantage of utilizing molecular docking in conjunction with sequence to structure fold prediction is that olfactory receptors that do not have a well characterized 3-dimensional structure can still be simulated and utilized in molecular docking computations, as long as the amino acid sequence of the given protein is known. Even in instances where the structure of a receptor protein has been measured, there may be an advantage to using structure prediction to further refine or validate the 3-dimensional structure to then be utilized in conjunction with molecular docking prediction(s).

Multiple programs and algorithms currently exist to calculate various aspects of molecular docking. Each has their own benefits and drawbacks. In this application it is understood that the term ‘molecular docking’ encompasses, holistically, the problem of predicting the activity of a ligand to a protein receptor. Thus, molecular docking as herein defined may include but is not limited to the use of computations for the following aspects of the problem: sequence to structure prediction, binding pocket identification, comparison with structure or activity of known protein sequences, calculation of most probable conformations of a given ligand, calculation of the most probable conformations of a given protein, docking of a ligand onto protein, prediction of binding affinity of the ligand-protein complex, prediction of likelihood of a given conformation of a ligand, protein, or ligand-protein complex, and other related calculations. Importantly, these calculations related to different aspects of the problem are interlinked, with the calculation of one aspect taking into account the results of the calculations of other aspects. Several algorithms, tools and techniques related to these calculations include: AutoDock including Autodock Vina, DiffDock, Rosetta Docking Protocol, MOE-Dock, rDock, Schodinger GLIDE, ProBIS, MetaPocket, PDBSiteScan, and more generally ensemble docking, free energy perturbation, inverse folding, reverse electron density screening, among many others. It is understood that the term ‘molecular docking’ or ‘in silico molecular docking’ herein is not limited to the use of any particular program or algorithm or set of programs or algorithms. Molecular docking in the context of this embodiment of the invention includes the utilization of these various computational tools to solve and/or elucidate the problem of predicting the activity of a given ligand or set of ligands against a given protein or set of proteins, and developing ligands with an optimized activity (including antagonistic or ‘blocking’ activity) against a given receptor or set of receptors.

This novel use and embodiment of the invention may be directed towards optimizing activity towards a specific olfactory receptor, as in the case of 2-{2-[3-(propan-2-yl)phenyl]propyl}-1,3-dioxan-5-one to VN1R1 as disclosed herein. Alternatively, and more broadly, this novel use of molecular docking simulations may be applied to the olfactory system as a whole, as a method of decoding the combinatorial sensory and neurological coding of said system. For example, this novel method may include the in silico, high-throughput testing of a given ligand against multiple receptors, or of all known receptors, to predict and codify its olfactory activity profile with regard to the array of receptors. It may also include the docking of an array of many olfactory ligands to each receptor in the olfactory system in order to codify that receptor's particular characteristic olfactory molecule response activity profile.

Once the response activity profile for all receptor sequences has been generated, the activity of a molecule or mixture can be codified across the entirety of the olfactory system. This, in turn then allows for the ‘compression’ and ‘replication’ of olfactory sensory experiences. Notably, this replication can potentially happen by utilizing molecules that are not contained in a given original olfactory mixture that is being replicated—for example by utilizing molecules or mixtures of molecules that are known or calculated to elicit the same pattern of olfactory receptor response as the original olfactory mixture. That in turn allows for a more universal scent and flavor replication system or device that utilizes a smaller number of individual scented components or ‘modules’. With a fully codified understanding of the olfactory system as in the case of this embodiment of the invention, it is possible to replicate any scent or flavor (as well as produce totally new scents and flavors) using a single system with a relatively small number of scent elements. These scent modules or scent elements can be thought of analogously as being similar to the vast array of different visible colors. In a similar manner to the way that the ‘RGB’ system can encode the range of human color vision, a set of scent modules developed with the embodiment disclosed herein could act as the RGB of the realm of human olfactory experience. This could also include flavor experience when the olfactory retronasal aspect of flavor is combined with taste, and would thus create a considerable advantage in being able to replicate any flavor with a given device that utilizes this smaller subset of olfactory and taste modules.

In one additional embodiment of the invention, the in silico molecular docking tools for predicting ligand activity on an olfactory receptor is screened using human sensory feedback. This includes for example the use of a trained panel to correlate the predicted activity with a particular sensory facet. As in the case of the development of an optimized ligand for the VN1R1 receptor and noting of a corresponding increase in pleasantness, fullness and complexity in the resulting optimized olfactory molecule, this technique can be utilized towards other facets. Unlike pharmaceutical development which requires time consuming and costly assays to validate the activity of a ligand on a receptor and subsequently use this information to further iterate on a candidate, the use of sensory feedback as an ‘assay’ can rapidly and much more affordably produce feedback that can be used for further iteration and optimization.

Importantly this combination of in silico prediction and sensory feedback screening, can be utilized towards the problem, previously mentioned, of decoding the human olfactory system. For example, using an iterative approach where the in-silico results are tested and validated with the sensory results and vis versa, particular olfactory facets can be disambiguated from others and correlated with the activity of certain receptors.

Within this process, olfactory sensory facets can be given new descriptors (potentially, novel terms that have not been previously applied to the sense of smell) that correspond to the activity of a particular receptor, or set of receptors. In the case of a facet that is perfectly correlated with a given olfactory receptor, this facet can be considered analogously to a primary color in the field of color vision, whereas distinct facets that arise from a combination of two or more receptors could be considered as a secondary or tertiary ‘color’. Candidate molecules or mixtures can be rated by a panel trained on a given set of facets and can thus be quickly screened and cross correlated with an in silico activity prediction. In silico prediction helps elucidate the connection between a given sensory facet and a given receptor's activity and creates a prediction to test using sensory feedback. The panel data in conjunction with the in silico predictions can together be used to create a system for decoding the entire olfactory system. This decoding can then form the basis for a device that can universally generate flavors or scents base on an efficient ‘compression’ that allows it to generate a large range of scents or flavors from a relatively small set of scent modules.

One surprising, additional aspect of this method of utilizing sensory feedback to correlate receptor activity with particular olfactory facets is that it can be used towards the development of therapeutic molecules. For example, molecules that are designed to target ectopically expressed olfactory receptors in the body (or indeed, olfactory sensory neurons in the olfactory epithelium as a therapeutic target) can be identified and/or made. Given that ectopically expressed olfactory receptors have the same amino acid sequence and respond to the same range of molecules as those in the olfactory system, a sensory panel (or trained individual) that is trained on a particular olfactory facet that has been correlated with the activity of a particular olfactory receptor, can gauge the level of activation of that receptor by how strongly a given olfactory molecule produces said olfactory facet. This in turn would allow the possibility of drug development cycles to be greatly accelerated, resulting in significant cost savings. For example, rather than running an assay for each new molecule candidate in an iterative drug development cycle, a well-trained panel could evaluate the strength of the smell of the molecule in a particular ‘direction’ or the strength of a particular facet within a molecule's olfactory profile. This can be rated for example by comparison to a reference standard with a known, agreed upon level of the facet, potentially in conjunction with detection threshold testing. Using this approach, several candidate molecules can be evaluated over the course of an hour and molecules that have a higher strength of a certain olfactory facet can then be further iterated upon or tested in an assay. It should be noted that this approach of utilizing sensory feedback to develop drug candidates can work in conjunction with traditional ‘wet lab’ assays, as well as or instead of an in silico approach. The novelty and particular usefulness of this method of drug discovery is that it can utilize human senses to greatly speed up development with the added benefit of reducing cost.

The methods disclosed herein of developing olfactory molecules via the utilization of in silico molecular docking (potentially also in conjunction with sensory feedback) can also be utilized towards developing the modules that a universal system would employ. For example, by developing a molecule that very selectively targets a particular receptor, this molecule can then be used as an olfactory module or primary olfactory ‘color ink’. The selectivity of the molecule allows for a greater efficiency when used as a module in that it will produce the desired scent or flavor facet without ‘crosstalk’ or ‘off target’ effects. This selectivity will allow a greater number of scents or flavors to be efficiently generated from a smaller number of modules. Given that the human olfactory system has approximately 400 receptors, the full range of human olfaction should be reproducible from 400 or less, well-tuned olfactory modules.

Separately from individual molecules developed or screened for use as a flavor or scent module in such a system, mixtures of molecules can be used as a module. In particular, molecules that share a structural and olfactory similarity can be blended together to target a particular facet that they have in common, while blending-out or ‘blurring’ their off-target facets. In addition to screening and inclusion on the basis of structural similarity, reverse electron density screening and other methods that take into account the location of charges within a molecular structure, separately from its structure, may also be employed.

### Selection of Additional Example Embodiments

Among the compounds of which this invention consists are the following list of compounds. This list is understood to be non-exhaustive. Chemical structure representations for the compounds below are included elsewhere within this application.
2-{2-[3-(propan-2-yl)phenyl]propyl}-1,3-dioxan-5-one
2-{2-[3-(propan-2-yl)phenyl]propyl}-1,3-dioxolane-4-carbaldehyde
2-[(2-bromophenyl)methyl]-1,3-dioxan-5-one
2-[(2,2,3-trimethylcyclopent-3-en-1-yl)methyl]-1,3-dioxane-5-carbaldehyde
2-[(2-bromophenyl)methyl]-1,3-dioxolane-4-carbaldehyde
2-[(2,4,6-tribromophenyl)methyl]-1,3-dioxan-5-one
2-[(2,4,6-tribromophenyl)methyl]-1,3-dioxolane-4-carbaldehyde
2-[(4-bromophenyl)methyl]-1,3-dioxan-5-one
2-[(4-bromophenyl)methyl]-1,3-dioxolane-4-carbaldehyde
2-[(4-tert-butylphenyl)methyl]-1,3-dioxan-5-one
2-[(4-tert-butylphenyl)methyl]-1,3-dioxolane-4-carbaldehyde
2-(2,2,3,3-tetramethylcyclopropyl)-1,3-dioxan-5-one
2-[(2,2,3-trimethylcyclopent-3-en-1-yl)methyl]-1,3-dioxan-5-one
5-methyl-2-[(2,2,3-trimethylcyclopent-3-en-1-yl)methyl]-1,3-dioxane-5-carbaldehyde
2-[(2,2,3-trimethylcyclopent-3-en-1-yl)methyl]-1,3-dioxolane-4-carbaldehyde
8,8,10,10-tetramethyl-1,5-dioxaspiro[5.5]undecane-3-carbaldehyde
3,8,8,10,10-pentamethyl-1,5-dioxaspiro[5.5]undecane-3-carbaldehyde
8,8,10,10-tetramethyl-1,5-dioxaspiro[5.5]undecan-3-one
7,7,9,9-tetramethyl-1,4-dioxaspiro[4.5]decane-2-carbaldehyde
2-(2,4-dimethylcyclohex-3-en-1-yl)-1,3-dioxan-5-one
2-(2,4-dimethylcyclohex-3-en-1-yl)-1,3-dioxolane-4-carbaldehyde
2-(2,4-dimethylcyclohex-3-en-1-yl)-1,3-dioxane-5-carbaldehyde
2-(2,4-dimethylcyclohex-3-en-1-yl)-5-methyl-1,3-dioxane-5-carbaldehyde
2-[(2,6,6-trimethylcyclohex-1-en-1-yl)methyl]-1,3-dioxan-5-one
2-[(2,6,6-trimethylcyclohex-1-en-1-yl)methyl]-1,3-dioxolane-4-carbaldehyde
2-[(2,6,6-trimethylcyclohex-1-en-1-yl)methyl]-1,3-dioxane-5-carbaldehyde
5-methyl-2-[(2,6,6-trimethylcyclohex-1-en-1-yl)methyl]-1,3-dioxane-5-carbaldehyde
2-(adamantan-1-yl)-1,3-dioxan-5-one
2-(adamantan-1-yl)-1,3-dioxolane-4-carbaldehyde
2-(adamantan-1-yl)-1,3-dioxane-5-carbaldehyde
2-(adamantan-1-yl)-5-methyl-1,3-dioxane-5-carbaldehyde
2-(3,3-dimethylcyclohexyl)-2-methyl-1,3-dioxan-5-one
2-(3,3-dimethylcyclohexyl)-2-methyl-1,3-dioxolane-4-carbaldehyde
2-(3,3-dimethylcyclohexyl)-2-methyl-1,3-dioxane-5-carbaldehyde
2-(3,3-dimethylcyclohexyl)-2,5-dimethyl-1,3-dioxane-5-carbaldehyde
2-[4-(tert-butoxy)phenyl]-1,3-dioxan-5-one
2-[4-(tert-butoxy)phenyl]-1,3-dioxolane-4-carbaldehyde
2-[4-(tert-butoxy)phenyl]-1,3-dioxane-5-carbaldehyde
2-[4-(tert-butoxy)phenyl]-5-methyl-1,3-dioxane-5-carbaldehyde
2-{4-[(trimethylsilyl)oxy]phenyl}-1,3-dioxan-5-one
2-{4-[(trimethylsilyl)oxy]phenyl}-1,3-dioxolane-4-carbaldehyde
2-{4-[(trimethylsilyl)oxy]phenyl}-1,3-dioxane-5-carbaldehyde
5-methyl-2-{4-[(trimethylsilyl)oxy]phenyl}-1,3-dioxane-5-carbaldehyde
2-({4-[(trimethylsilyl)oxy]phenyl}methyl)-1,3-dioxan-5-one
2-({4-[(trimethylsilyl)oxy]phenyl}methyl)-1,3-dioxolane-4-carbaldehyde
2-({4-[(trimethylsilyl)oxy]phenyl}methyl)-1,3-dioxane-5-carbaldehyde
5-methyl-2-({4-[(trimethylsilyl)oxy]phenyl}methyl)-1,3-dioxane-5-carbaldehyde
2-[(2-methylphenyl)methyl]-1,3-dioxan-5-one
2-[(2-methylphenyl)methyl]-1,3-dioxolane-4-carbaldehyde
2-[(2-methylphenyl)methyl]-1,3-dioxane-5-carbaldehyde
5-methyl-2-[(2-methylphenyl)methyl]-1,3-dioxane-5-carbaldehyde
2-{1-[4-(2-methylpropyl)phenyl]ethyl}-1,3-dioxan-5-one
2-{1-[4-(2-methylpropyl)phenyl]ethyl}-1,3-dioxolane-4-carbaldehyde
2-[1-(4-ethylphenyl)ethyl]-1,3-dioxan-5-one
2-[1-(4-ethylphenyl)ethyl]-1,3-dioxolane-4-carbaldehyde
2-[1-(4-ethylphenyl)ethyl]-1,3-dioxane-5-carbaldehyde
2-[1-(4-ethylphenyl)ethyl]-5-methyl-1,3-dioxane-5-carbaldehyde
2-(2,6,6-trimethylcyclohexa-1,3-dien-1-yl)-1,3-dioxan-5-one
2-(2,6,6-trimethylcyclohexa-1,3-dien-1-yl)-1,3-dioxolane-4-carbaldehyde
2-(2,6,6-trimethylcyclohexa-1,3-dien-1-yl)-1,3-dioxane-5-carbaldehyde
5-methyl-2-(2,6,6-trimethylcyclohexa-1,3-dien-1-yl)-1,3-dioxane-5-carbaldehyde
2-[(2,6,6-trimethylcyclohexa-1,3-dien-1-yl)methyl]-1,3-dioxan-5-one
2-[(2,6,6-trimethylcyclohexa-1,3-dien-1-yl)methyl]-1,3-dioxolane-4-carbaldehyde
2-[(2,6,6-trimethylcyclohexa-1,3-dien-1-yl)methyl]-1,3-dioxane-5-carbaldehyde
5-methyl-2-[(2,6,6-trimethylcyclohexa-1,3-dien-1-yl)methyl]-1,3-dioxane-5-carbaldehyde
2-[2-(2-bromophenyl)propyl]-1,3-dioxan-5-one
2-[2-(2-bromophenyl)propyl]-1,3-dioxolane-4-carbaldehyde
2-[1-(2-bromophenyl)ethyl]-1,3-dioxan-5-one
2-[1-(2-bromophenyl)ethyl]-1,3-dioxolane-4-carbaldehyde
2-[1-(2-bromophenyl)ethyl]-1,3-dioxane-5-carbaldehyde
2-[1-(2-bromophenyl)ethyl]-5-methyl-1,3-dioxane-5-carbaldehyde
2-[6-methyl-8-(propan-2-yl)bicyclo[2.2.2]oct-5-en-2-yl]-1,3-dioxan-5-one
2-[6-methyl-8-(propan-2-yl)bicyclo[2.2.2]oct-5-en-2-yl]-1,3-dioxolane-4-carbaldehyde
2-[6-methyl-8-(propan-2-yl)bicyclo[2.2.2]oct-5-en-2-yl]-1,3-dioxane-5-carbaldehyde
5-methyl-2-[6-methyl-8-(propan-2-yl)bicyclo[2.2.2]oct-5-en-2-yl]-1,3-dioxane-5-carbaldehyde
2-(2-{6,6-dimethylbicyclo[3.1.1]hept-2-en-2-yl}ethyl)-1,3-dioxan-5-one
2-(2-{6,6-dimethylbicyclo[3.1.1]hept-2-en-2-yl}ethyl)-1,3-dioxolane-4-carbaldehyde
2-(2-{6,6-dimethylbicyclo[3.1.1]hept-2-en-2-yl}ethyl)-1,3-dioxane-5-carbaldehyde
2-(2-{6,6-dimethylbicyclo[3.1.1]hept-2-en-2-yl}ethyl)-5-methyl-1,3-dioxane-5-carbaldehyde
2-(2,4,6-trimethylcyclohex-3-en-1-yl)-1,3-dioxan-5-one
2-(2,4,6-trimethylcyclohex-3-en-1-yl)-1,3-dioxolane-4-carbaldehyde
2-(2,4,6-trimethylcyclohex-3-en-1-yl)-1,3-dioxane-5-carbaldehyde
5-methyl-2-(2,4,6-trimethylcyclohex-3-en-1-yl)-1,3-dioxane-5-carbaldehyde
2-(2,2,3,3-tetramethylcyclopropyl)-1,3-dioxolane-4-carbaldehyde
2-(2,2,3,3-tetramethylcyclopropyl)-1,3-dioxolane-4-carbaldehyde
5-methyl-2-(2,2,3,3-tetramethylcyclopropyl)-1,3-dioxane-5-carbaldehyde
2-[2-(2,2,3,3-tetramethylcyclopropyl)-1,3-dioxolan-4-yl]acetaldehyde
ethyl 2-formyl-1,4-dioxaspiro[4.5]decane-7-carboxylate
ethyl 2-(2-oxoethyl)-1,4-dioxaspiro[4.5]decane-7-carboxylate
ethyl 3-oxo-1,5-dioxaspiro[5.5]undecane-8-carboxylate
7-methyl-6-[(2Z)-pent-2-en-1-yl]-1,4-dioxaspiro[4.4]non-6-ene-2-carbaldehyde
2-{7-methyl-6-[(2Z)-pent-2-en-1-yl]-1,4-dioxaspiro[4.4]non-6-en-2-yl}acetaldehyde
2-methyl-1-[(2Z)-pent-2-en-1-yl]-6,10-dioxaspiro[4.5]dec-1-en-8-one
2-(4-hydroxy-3-methoxyphenyl)-1,3-dioxan-5-one
2-(4-hydroxy-3-methoxyphenyl)-1,3-dioxolane-4-carbaldehyde
2-(4-hydroxy-3-methoxyphenyl)-1,3-dioxane-5-carbaldehyde
2-(4-hydroxy-3-methoxyphenyl)-5-methyl-1,3-dioxane-5-carbaldehyde
2-[2-(4-hydroxy-3-methoxyphenyl)-1,3-dioxolan-4-yl]acetaldehyde
2-(3-ethoxy-4-hydroxyphenyl)-1,3-dioxan-5-one
2-(3-ethoxy-4-hydroxyphenyl)-1,3-dioxolane-4-carbaldehyde
2-(2-bromophenyl)-1,4-dioxepan-6-one
2-[(2-bromophenyl)methyl]-1,4-dioxepan-6-one
2-[(2-bromo-5-methylphenyl)methyl]-1,4-dioxepan-6-one
2-({2-bromo-4-[(1E)-3,3-dimethylbut-1-en-1-yl]phenyl}methyl)-1,4-dioxepan-6-one
2-{2-bromo-5-[(1E)-prop-1-en-1-yl]phenyl}-1,4-dioxepan-6-one
2-{2-[(2,2,3-trimethylcyclopent-3-en-1-yl)methyl]-1,3-dioxolan-4-yl}acetaldehyde
2-[(4,4-dimethyl-3,4-dihydronaphthalen-2-yl)methyl]-1,3-dioxan-5-one
2-[(4-methyl-3,4-dihydronaphthalen-1-yl)methyl]-1,3-dioxan-5-one
2-(4,4-dimethyl-3,4-dihydronaphthalen-2-yl)-1,3-dioxan-5-one
2-[2-(4,4-dimethyl-3,4-dihydronaphthalen-2-yl)ethyl]-1,3-dioxan-5-one
2-(1,2,3,4-tetrahydronaphthalen-2-yl)-1,3-dioxan-5-one
2-[(naphthalen-2-yl)methyl]-1,3-dioxan-5-one
2-(naphthalen-2-yl)-1,3-dioxan-5-one
2-(4-methyl-1,2-dihydronaphthalen-2-yl)-1,3-dioxan-5-one
2-(naphthalen-1-yl)-1,3-dioxan-5-one
2-[2-(naphthalen-1-yl)-1,3-dioxolan-4-yl]acetaldehyde
2-(3,4-dihydronaphthalen-2-yl)-1,3-dioxan-5-one
2-(3,4-dihydronaphthalen-2-yl)-1,3-dioxolan-4-one
2-(3,4-dihydronaphthalen-2-yl)-5,5-dimethyl-1,3-dioxolan-4-one
2-(2-{3-[(trimethylsilyl)oxy]phenyl}propyl)-1,3-dioxan-5-one
2-(2-{3-[(trimethylsilyl)oxy]phenyl}propyl)-1,3-dioxolane-4-carbaldehyde
2-(2H-1,3-benzodioxol-4-yl)-1,3-dioxan-5-one
2-(2H-1,3-benzodioxol-4-yl)-1,3-dioxolane-4-carbaldehyde
1-{2H,3aH,8H,8aH-indeno[1,2-d][1,3]dioxol-2-yl}ethan-1-one
2-methyl-2H,3aH,8H,8aH-indeno[1,2-d][1,3]dioxole-2-carbaldehyde
2-[(2,3-dihydro-1-benzofuran-5-yl)methyl]-1,3-dioxan-5-one
2-(1-{2-bromo-5-[(2Z)-hex-2-en-1-yl]phenyl}ethyl)-1,3-dioxan-5-one
2-(1-{2-bromo-5-[(2Z)-hex-2-en-1-yl]phenyl}ethyl)-1,3-dioxolane-4-carbaldehyde
2-(2-{[3-(3-hydroxy-3-methylbutan-2-yl)-2,2-dimethylcyclopent-3-en-1-yl]methyl}-1,3-dioxolan-4-yl)acetaldehyde
2-{[3-(3-hydroxy-3-methylbutan-2-yl)-2,2-dimethylcyclopent-3-en-1-yl]methyl}-1,3-dioxan-5-one

FIG. 1 is the molecule 2-{2-[3-(propan-2-yl)phenyl]propyl}-1,3-dioxan-5-one (Example 1 in this document) docked in the VN1R1 receptor (human vomeronasal 1 receptor 1) using predictive docking and protein folding software algorithms. The protein structure of the receptor is rendered at 50% opacity with the ligand 2-{2-[3-(propan-2-yl)phenyl]propyl}-1,3-dioxan-5-one is at 100% opacity and is indicated by a black circle. Four separate views are shown of the complex at different angles of rotation.

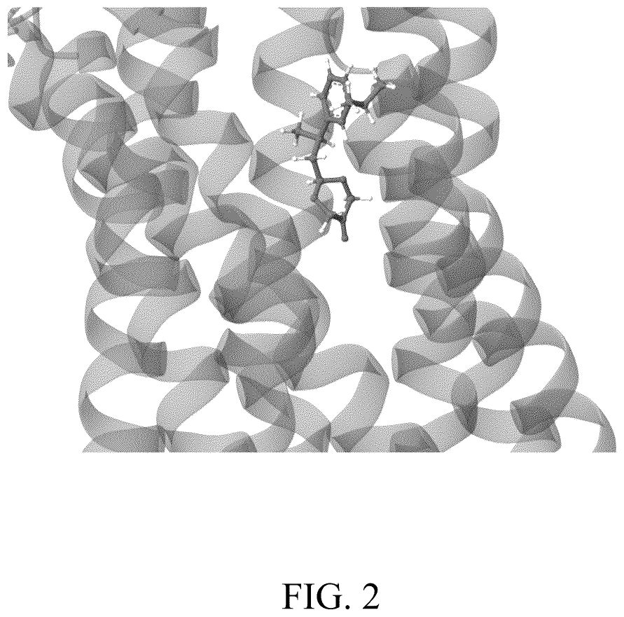

FIG. 2 is a close up view of 2-{2-[3-(propan-2-yl)phenyl]propyl}-1,3-dioxan-5-one (Example 1) docked in the VN1R1 receptor.

FIG. 3 is a hydrogen bond shown between the ketone group of 2-{2-[3-(propan-2-yl)phenyl]propyl}-1,3-dioxan-5-one (Example 1) and the —OH group of the Tyrosine residue #268 on the protein VN1R1. The interaction is indicated with a black circle around the participating groups and a dotted line between them.

FIG. 4 is a hydrogen bond shown between the ether group of 2-{2-[3-(propan-2-yl)phenyl]propyl}-1,3-dioxan-5-one (Example 1) and the OH group of the Tyrosine residue #224 on the protein VN1R1. The interaction is indicated with a black circle around the participating groups and a dotted line between them.

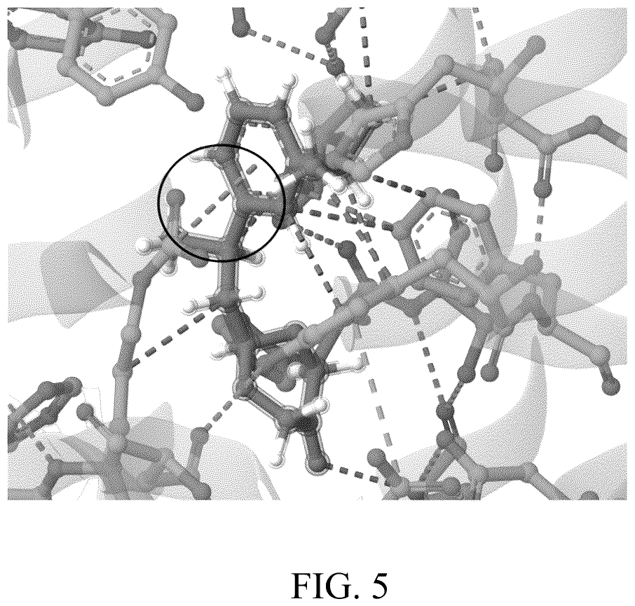

FIG. 5 is a cation-pi interaction shown between the benzene ring of 2-{2-[3-(propan-2-yl)phenyl]propyl}-1,3-dioxan-5-one (Example 1) and the guanidine group of the arginine residue #129 on the protein VN1R1. The interaction is indicated with a black circle around the participating groups and a dotted line between them.

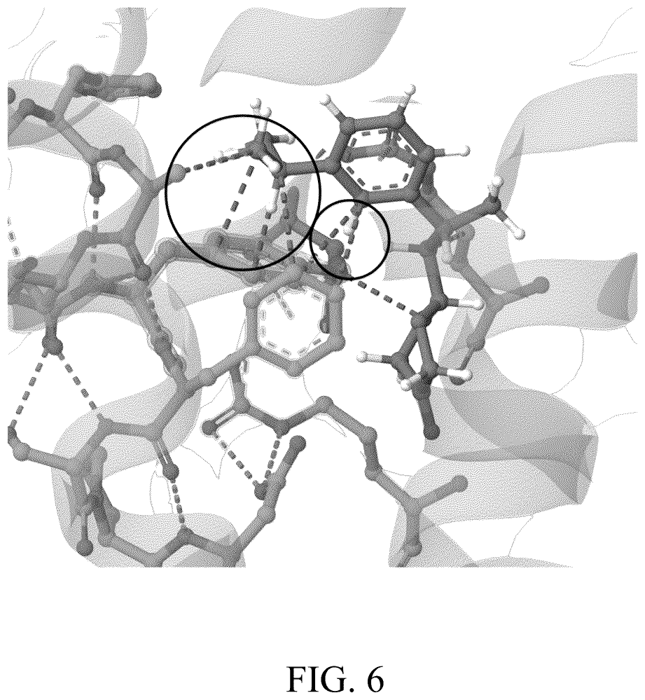

FIG. 6 are hydrophobic interactions shown between the isopropyl tail and benzene ring of 2-{2-[3-(propan-2-yl)phenyl]propyl}-1,3-dioxan-5-one (Example 1) with various residues of VN1R1. The interactions are indicated with black circles around the participating groups and dotted lines between them. Other hydrophobic interactions are predicted but not shown in this image.

### Additional Example Embodiments and Structure Images

The embodiments detailed below show the range of compounds. These embodiments are a non-exhaustive list of the compounds that have the properties as disclosed herein.

### Example 6

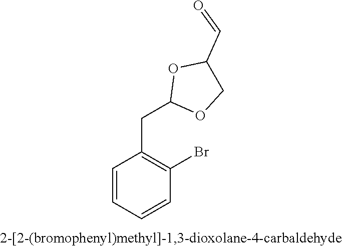 [SMILES: O=CC1COC(Cc2ccccc2Br)O1]

### Example 7

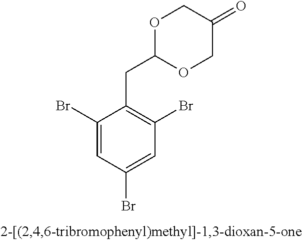 [SMILES: O=C1COC(Cc2c(Br)cc(Br)cc2Br)OC1]

### Example 8

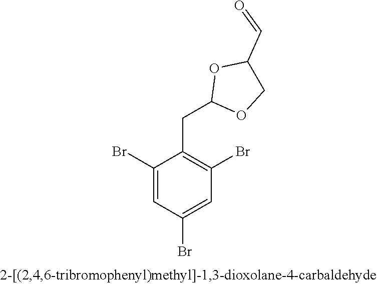 [SMILES: O=CC1COC(Cc2c(Br)cc(Br)cc2Br)O1]

### Example 9

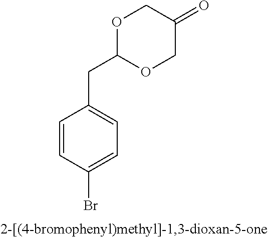 [SMILES: O=C1COC(Cc2ccc(Br)cc2)OC1]

### Example 10

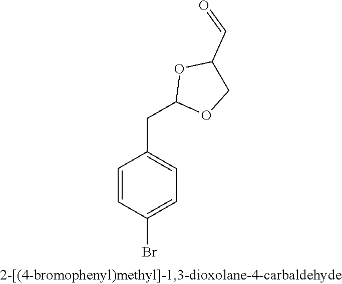 [SMILES: O=CC1COC(Cc2ccc(Br)cc2)O1]

### Example 11

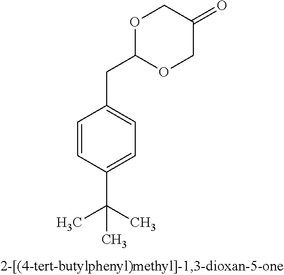 [SMILES: CC(C)(C)c1ccc(CC2OCC(=O)CO2)cc1]

### Example 12

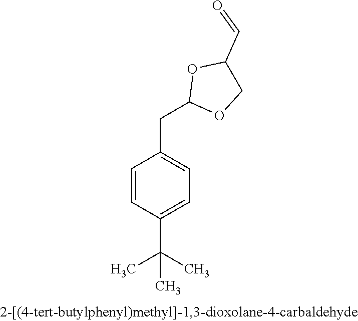 [SMILES: CC(C)(C)c1ccc(CC2OCC(C=O)O2)cc1]

### Example 13

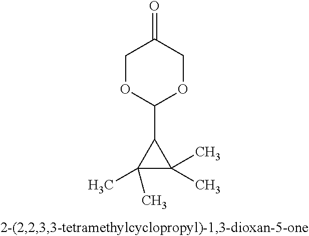

### Example 14

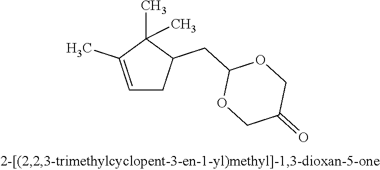 [SMILES: CC1=CCC(CC2OCC(=O)CO2)C1(C)C]

### Example 15

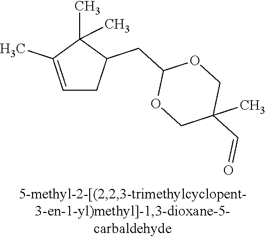

### Example 16

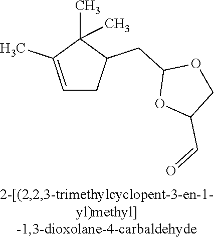 [SMILES: CC1=CCC(CC2OCC(C=O)O2)C1(C)C]

### Example 17

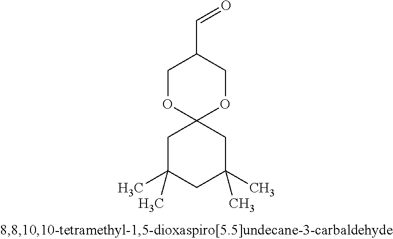

### Example 18

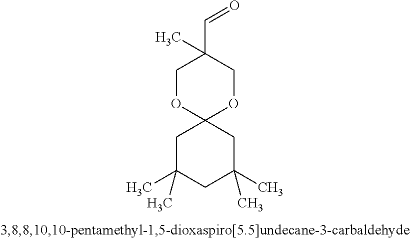

### Example 19

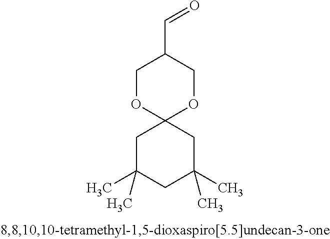

### Example 20

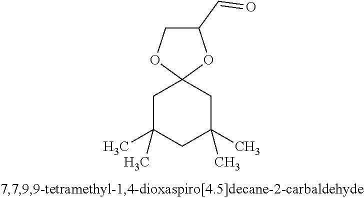 [SMILES: CC1(C)CC(C)(C)CC2(C1)OCC(C=O)O2]

### Example 21

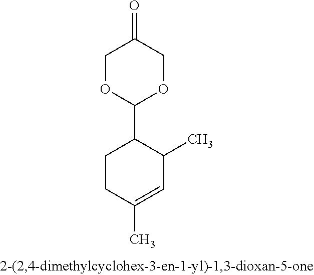

### Example 22

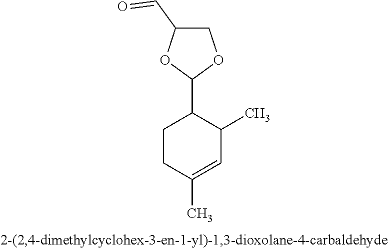

### Example 23

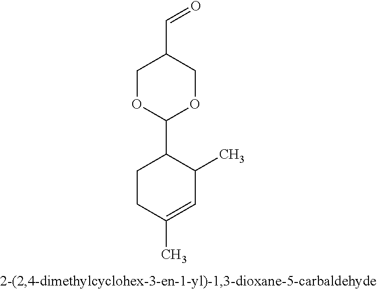 [SMILES: CC1=CC(C)C(C2OCC(C=O)CO2)CC1]

### Example 24

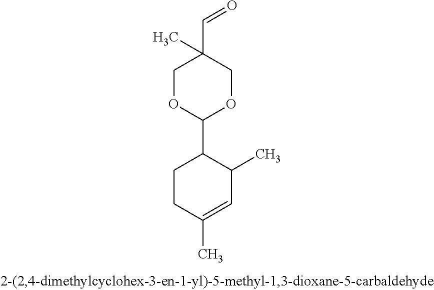 [SMILES: CC1=CC(C)C(C2OCC(C)(C=O)CO2)CC1]

### Example 25

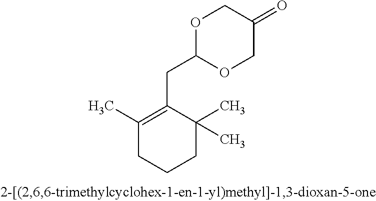 [SMILES: CC1=C(CC2OCC(=O)CO2)C(C)(C)CCC1]

### Example 26

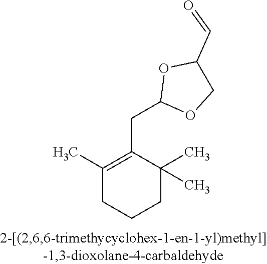 [SMILES: CC1=C(CC2OCC(C=O)O2)C(C)(C)CCC1]

### Example 27

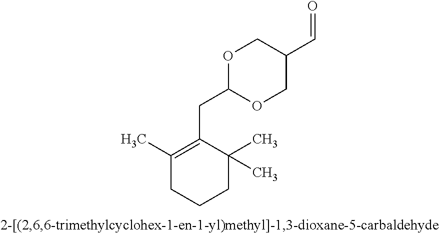 [SMILES: CC1=C(CC2OCC(C=O)CO2)C(C)(C)CCC1]

### Example 28

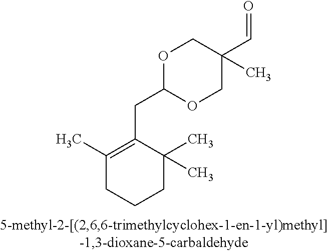

### Example 29

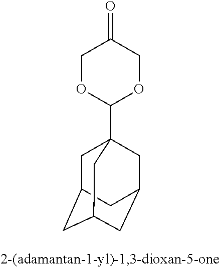 [SMILES: O=C1COC(C23CC4CC(CC(C4)C2)C3)OC1]

### Example 30

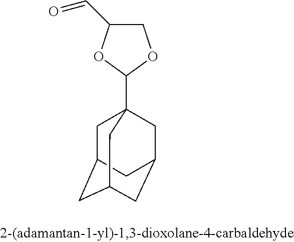 [SMILES: O=CC1COC(C23CC4CC(CC(C4)C2)C3)O1]

### Example 31

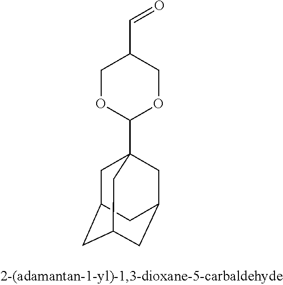 [SMILES: O=CC1COC(C23CC4CC(CC(C4)C2)C3)OC1]

### Example 32

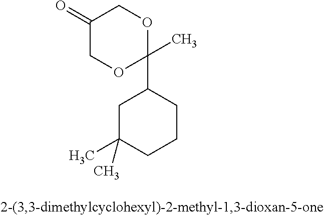 [SMILES: CC1(C)CCCC(C2(C)OCC(=O)CO2)C1]

### Example 33

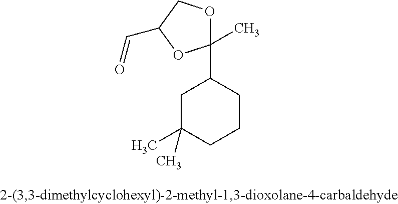 [SMILES: CC1(C)CCCC(C2(C)OCC(C=O)O2)C1]

### Example 34

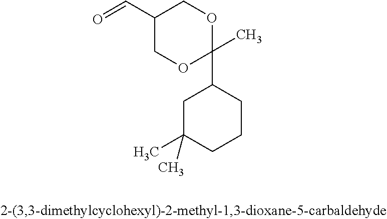 [SMILES: CC1(C)CCCC(C2(C)OCC(C=O)CO2)C1]

### Example 35

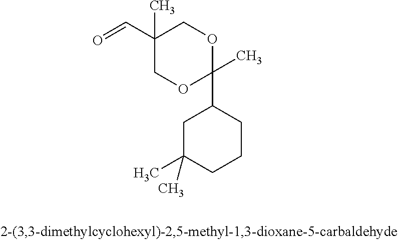 [SMILES: CC1(C)CCCC(C2(C)OCC(C)(C=O)CO2)C1]

### Example 36

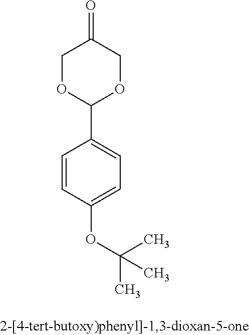 [SMILES: CC(C)(C)Oc1ccc(C2OCC(=O)CO2)cc1]

### Example 37

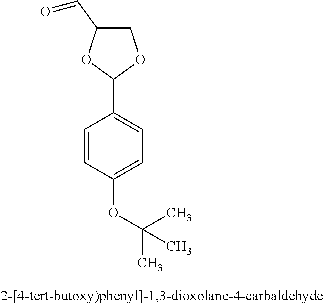 [SMILES: CC(C)(C)Oc1ccc(C2OCC(C=O)O2)cc1]

### Example 38

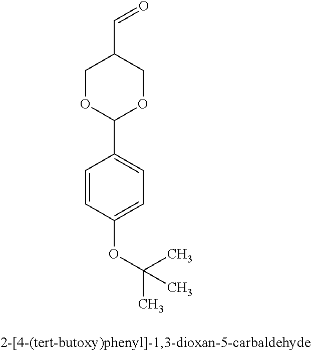 [SMILES: CC(C)(C)Oc1ccc(C2OCC(C=O)CO2)cc1]

### Example 39

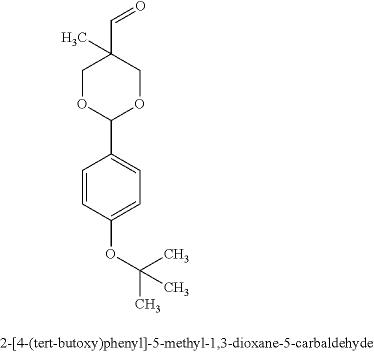 [SMILES: CC1(C=O)COC(c2ccc(OC(C)(C)C)cc2)OC1]

### Example 40

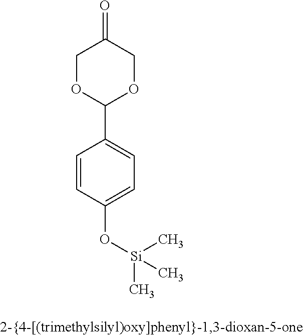 [SMILES: C[Si](C)(C)Oc1ccc(C2OCC(=O)CO2)cc1]

### Example 41

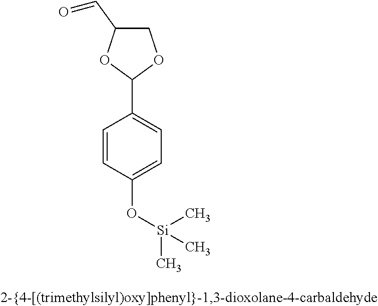 [SMILES: C[Si](C)(C)Oc1ccc(C2OCC(C=O)O2)cc1]

### Example 42

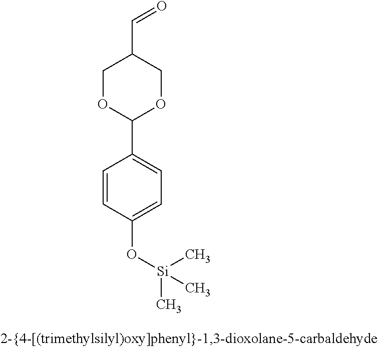 [SMILES: C[Si](C)(C)Oc1ccc(C2OCC(C=O)CO2)cc1]

### Example 43

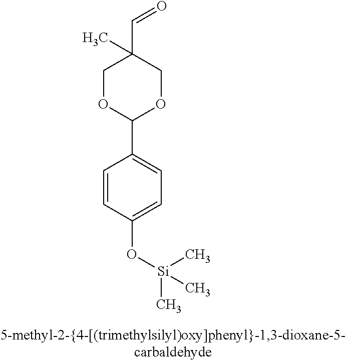 [SMILES: CC1(C=O)COC(c2ccc(O[Si](C)(C)C)cc2)OC1]

### Example 44

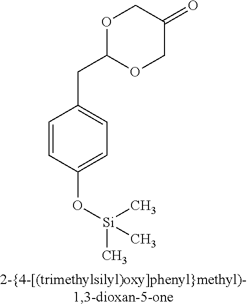 [SMILES: C[Si](C)(C)Oc1ccc(CC2OCC(=O)CO2)cc1]

### Example 45

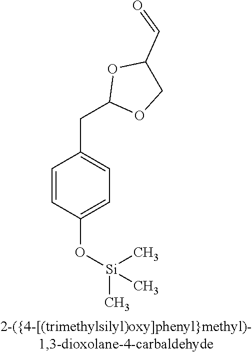 [SMILES: C[Si](C)(C)Oc1ccc(CC2OCC(C=O)O2)cc1]

### Example 46

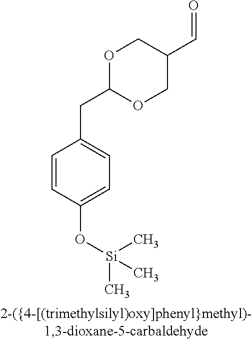 [SMILES: C[Si](C)(C)Oc1ccc(CC2OCC(C=O)CO2)cc1]

### Example 47

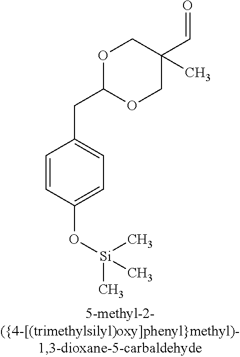 [SMILES: CC1(C=O)COC(Cc2ccc(O[Si](C)(C)C)cc2)OC1]

### Example 48

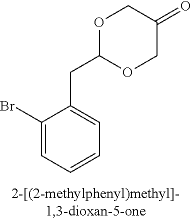 [SMILES: O=C1COC(Cc2ccccc2Br)OC1]

### Example 49

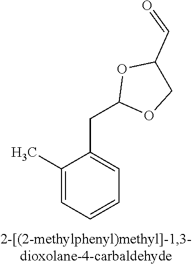

### Example 50

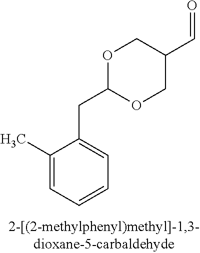 [SMILES: Cc1ccccc1CC1OCC(C=O)CO1]

### Example 51

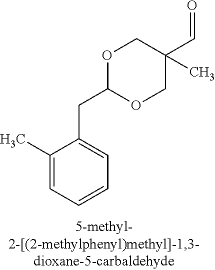

### Example 52

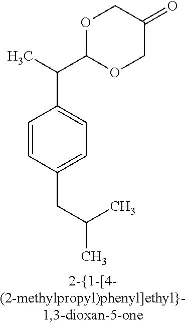 [SMILES: CC(C)Cc1ccc(C(C)C2OCC(=O)CO2)cc1]

### Example 53

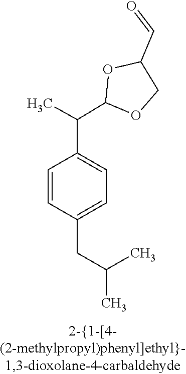 [SMILES: CC(C)Cc1ccc(C(C)C2OCC(C=O)O2)cc1]

### Example 54

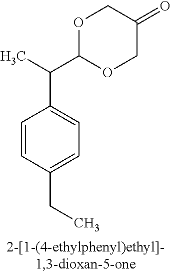 [SMILES: CCc1ccc(C(C)C2OCC(=O)CO2)cc1]

### Example 55

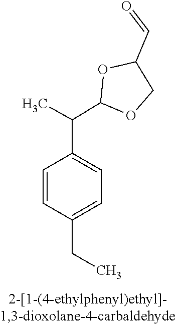 [SMILES: CCc1ccc(C(C)C2OCC(C=O)O2)cc1]

### Example 56

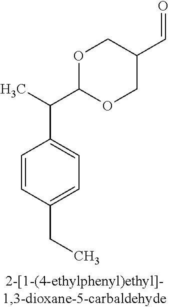 [SMILES: CCc1ccc(C(C)C2OCC(C=O)CO2)cc1]

### Example 57

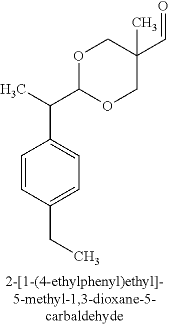 [SMILES: CCc1ccc(C(C)C2OCC(C)(C=O)CO2)cc1]

### Example 58

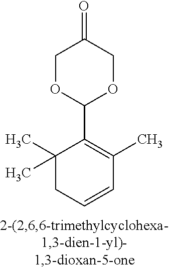

### Example 59

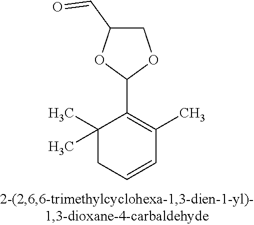 [SMILES: CC1=C(C2OCC(C=O)O2)C(C)(C)CC=C1]

### Example 60

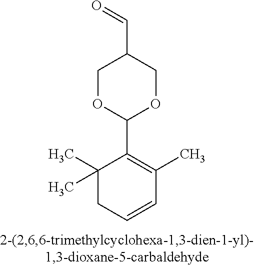

### Example 61

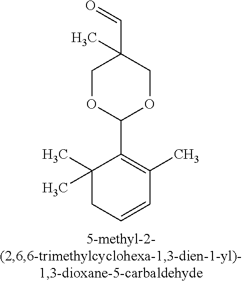

### Example 62

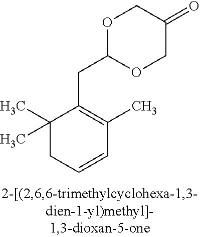 [SMILES: CC1=C(CC2OCC(=O)CO2)C(C)(C)CC=C1]

### Example 63

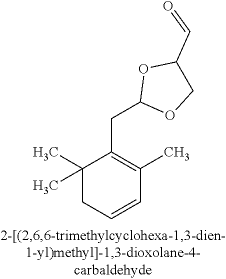 [SMILES: CC1=C(CC2OCC(C=O)O2)C(C)(C)CC=C1]

### Example 64

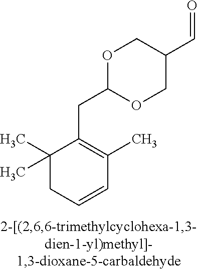 [SMILES: CC1=C(CC2OCC(C=O)CO2)C(C)(C)CC=C1]

### Example 65

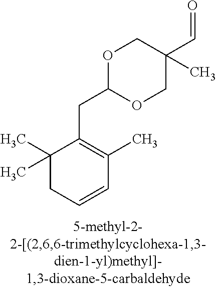

### Example 66

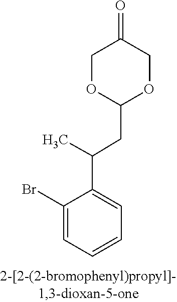 [SMILES: CC(CC1OCC(=O)CO1)c1ccccc1Br]

### Example 67

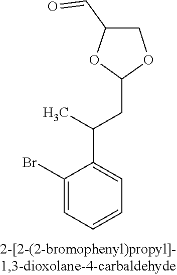 [SMILES: CC(CC1OCC(C=O)O1)c1ccccc1Br]

### Example 68

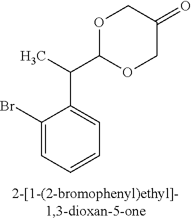 [SMILES: CC(c1ccccc1Br)C1OCC(=O)CO1]

### Example 69

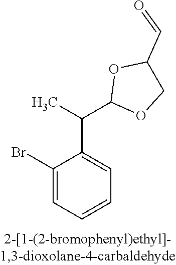 [SMILES: CC(c1ccccc1Br)C1OCC(C=O)O1]

### Example 70

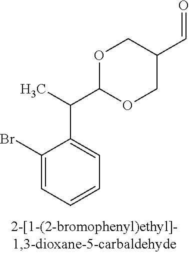 [SMILES: CC(c1ccccc1Br)C1OCC(C=O)CO1]

### Example 71

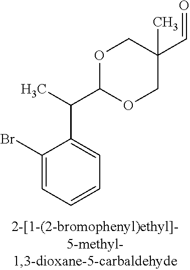 [SMILES: CC(c1ccccc1Br)C1OCC(C)(C=O)CO1]

### Example 72

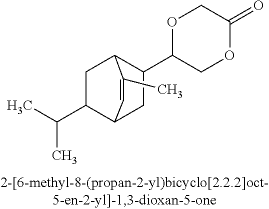 [SMILES: CC1=CC2CC(C3COC(=O)CO3)C1CC2C(C)C]

### Example 73

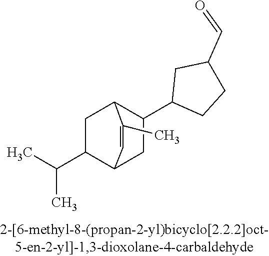 [SMILES: CC1=CC2CC(C3CCC(C=O)C3)C1CC2C(C)C]

### Example 74

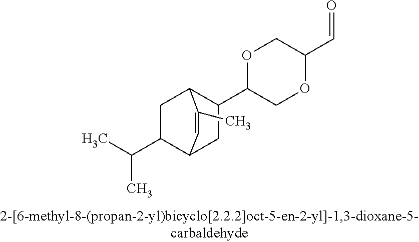 [SMILES: CC1=CC2CC(C3COC(C=O)CO3)C1CC2C(C)C]

### Example 75

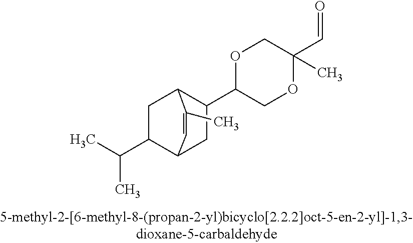 [SMILES: CC1=CC2CC(C3COC(C)(C=O)CO3)C1CC2C(C)C]

### Example 76

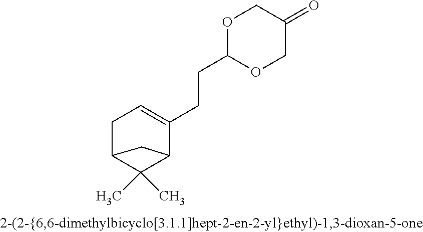 [SMILES: CC1(C)C2CC=C(CCC3OCC(=O)CO3)C1C2]

### Example 77

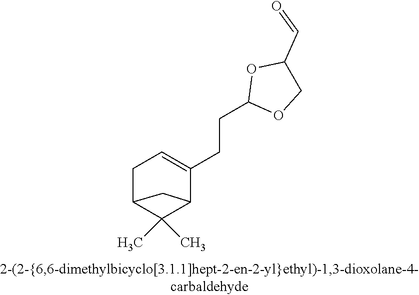 [SMILES: CC1(C)C2CC=C(CCC3OCC(C=O)O3)C1C2]

### Example 78

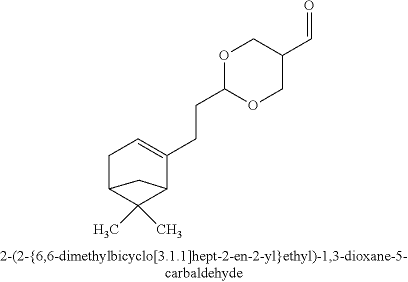 [SMILES: CC1(C)C2CC=C(CCC3OCC(C=O)CO3)C1C2]

### Example 79

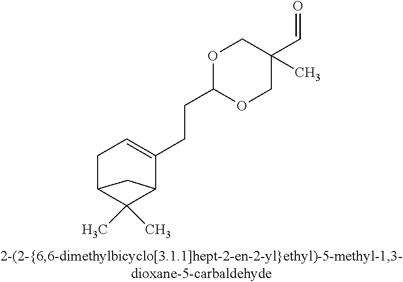 [SMILES: CC1(C=O)COC(CCC2=CCC3CC2C3(C)C)OC1]

### Example 80

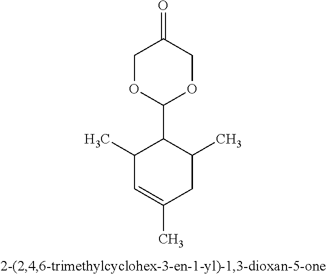

### Example 81

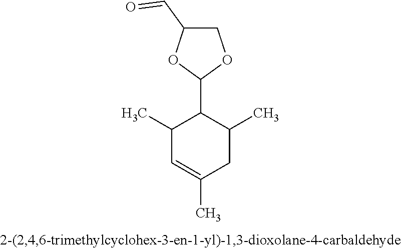 [SMILES: CC1=CC(C)C(C2OCC(C=O)O2)C(C)C1]

### Example 82

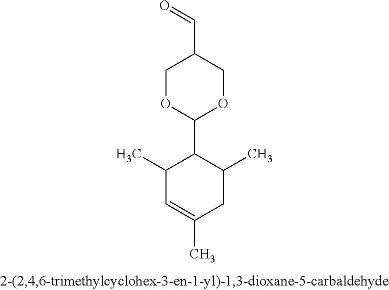

### Example 83

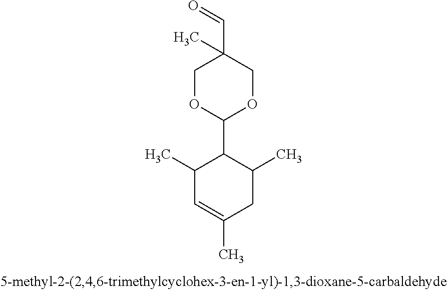

### Example 84

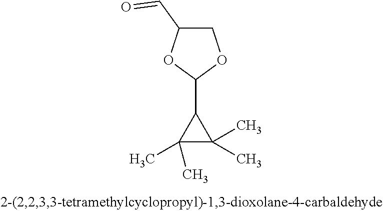

### Example 85

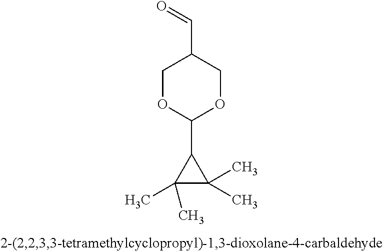

### Example 86

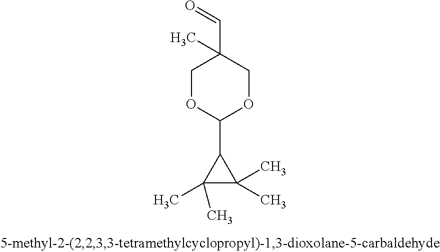

### Example 87

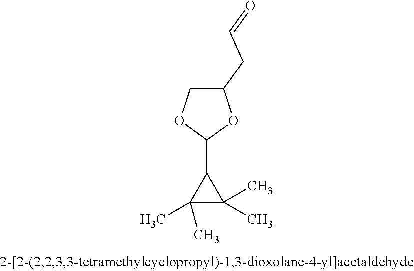

### Example 88

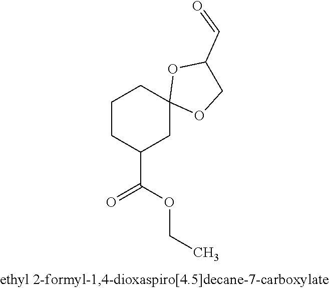 [SMILES: CCOC(=O)C1CCCC2(C1)OCC(C=O)O2]

### Example 89

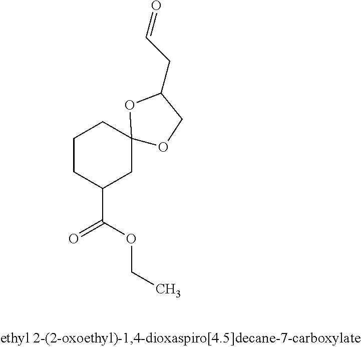 [SMILES: CCOC(=O)C1CCCC2(C1)OCC(CC=O)O2]

### Example 90

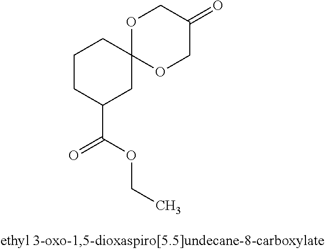 [SMILES: CCOC(=O)C1CCCC2(C1)OCC(=O)CO2]

### Example 91

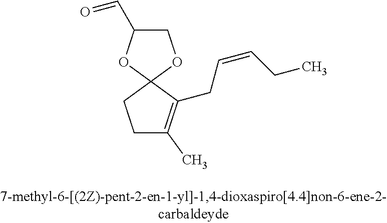 [SMILES: CC/C=C\CC1=C(C)CCC12OCC(C=O)O2]

### Example 92

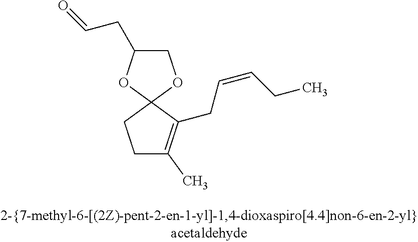 [SMILES: CC/C=C\CC1=C(C)CCC12OCC(CC=O)O2]

### Example 93

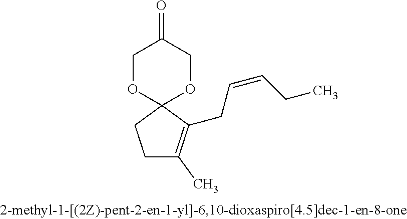 [SMILES: CC/C=C\CC1=C(C)CCC12OCC(=O)CO2]

### Example 94

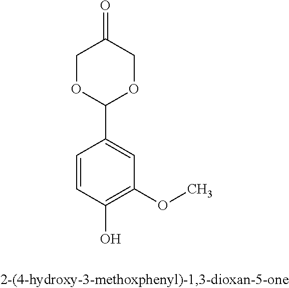 [SMILES: COc1cc(C2OCC(=O)CO2)ccc1O]

### Example 95

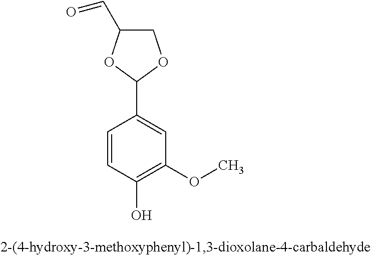 [SMILES: COc1cc(C2OCC(C=O)O2)ccc1O]

### Example 96

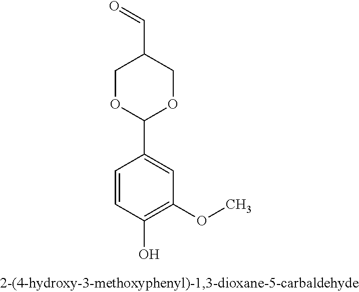 [SMILES: COc1cc(C2OCC(C=O)CO2)ccc1O]

### Example 97

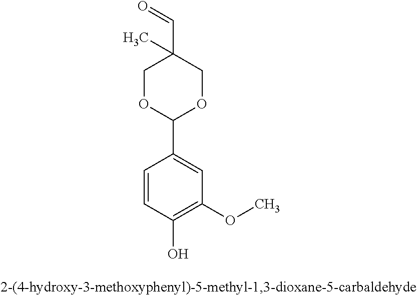 [SMILES: COc1cc(C2OCC(C)(C=O)CO2)ccc1O]

### Example 98

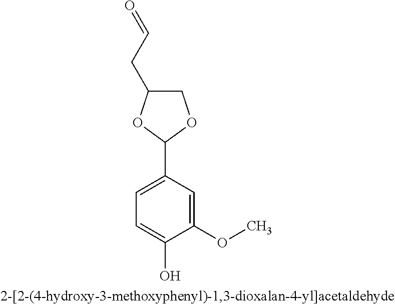 [SMILES: COc1cc(C2OCC(CC=O)O2)ccc1O]

### Example 99

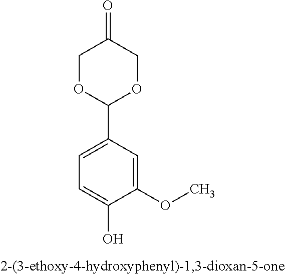 [SMILES: COc1cc(C2OCC(=O)CO2)ccc1O]

### Example 100

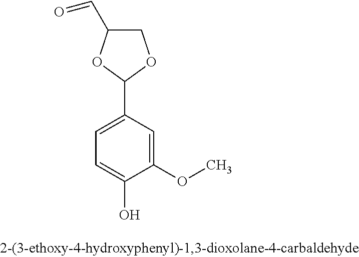 [SMILES: COc1cc(C2OCC(C=O)O2)ccc1O]

### Example 101

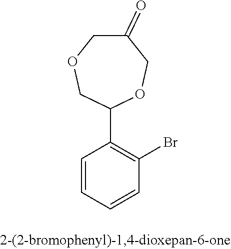 [SMILES: O=C1COCC(c2ccccc2Br)OC1]

### Example 102

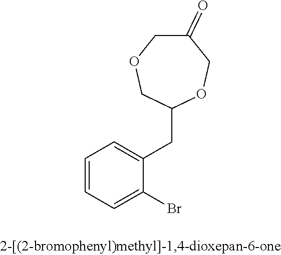 [SMILES: O=C1COCC(Cc2ccccc2Br)OC1]

### Example 103

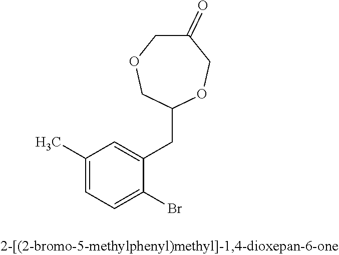 [SMILES: Cc1ccc(Br)c(CC2COCC(=O)CO2)c1]

### Example 104

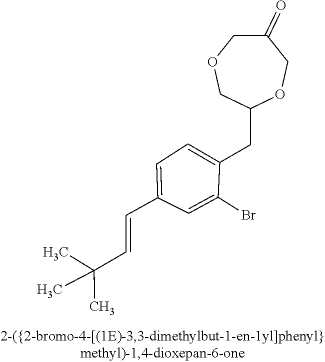 [SMILES: CC(C)(C)/C=C/c1ccc(CC2COCC(=O)CO2)c(Br)c1]

### Example 105

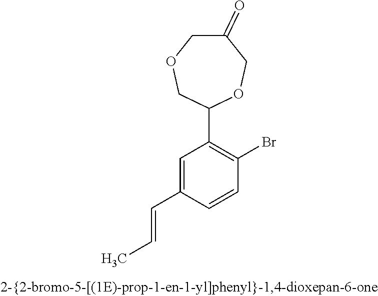 [SMILES: C/C=C/c1ccc(Br)c(C2COCC(=O)CO2)c1]

### Example 106

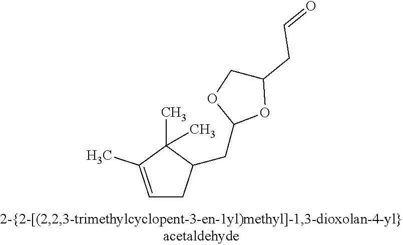 [SMILES: CC1=CCC(CC2OCC(CC=O)O2)C1(C)C]

### Example 107

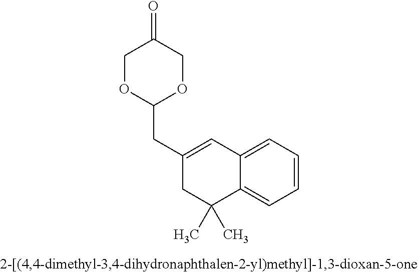 [SMILES: CC1(C)CC(CC2OCC(=O)CO2)=Cc2ccccc21]

### Example 108

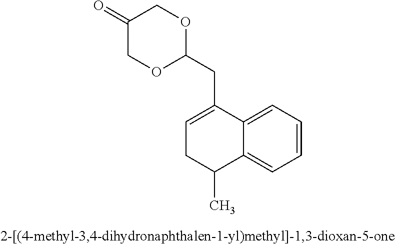 [SMILES: CC1CC=C(CC2OCC(=O)CO2)c2ccccc21]

### Example 109

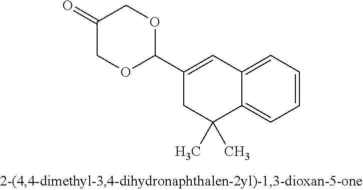 [SMILES: CC1(C)CC(C2OCC(=O)CO2)=Cc2ccccc21]

### Example 110

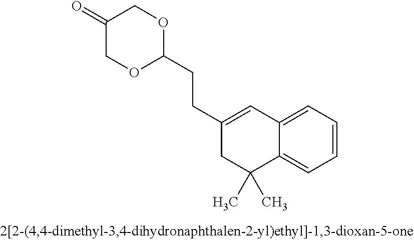 [SMILES: CC1(C)CC(CCC2OCC(=O)CO2)=Cc2ccccc21]

### Example 111

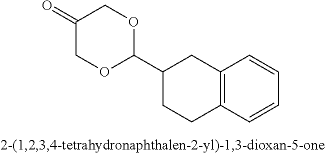 [SMILES: O=C1COC(C2CCc3ccccc3C2)OC1]

### Example 112

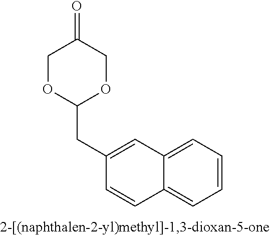 [SMILES: O=C1COC(Cc2ccc3ccccc3c2)OC1]

### Example 113

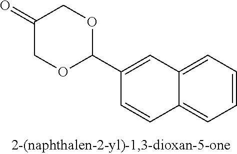 [SMILES: O=C1COC(c2ccc3ccccc3c2)OC1]

### Example 114

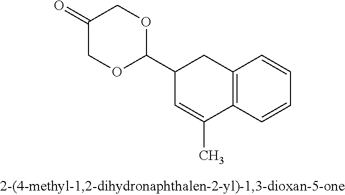 [SMILES: CC1=CC(C2OCC(=O)CO2)Cc2ccccc21]

### Example 115

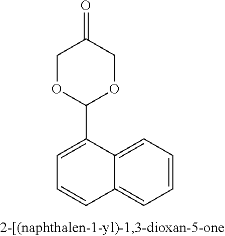 [SMILES: O=C1COC(c2cccc3ccccc23)OC1]

### Example 116

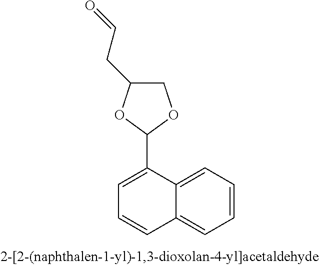 [SMILES: O=CCC1COC(c2cccc3ccccc23)O1]

### Example 117

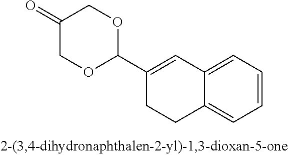 [SMILES: O=C1COC(C2=Cc3ccccc3CC2)OC1]

### Example 118

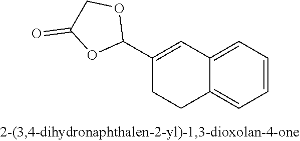 [SMILES: O=C1COC(C2=Cc3ccccc3CC2)O1]

### Example 119

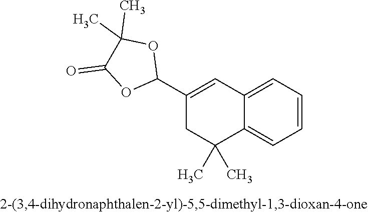 [SMILES: CC1(C)OC(C2=Cc3ccccc3C(C)(C)C2)OC1=O]

### Example 120

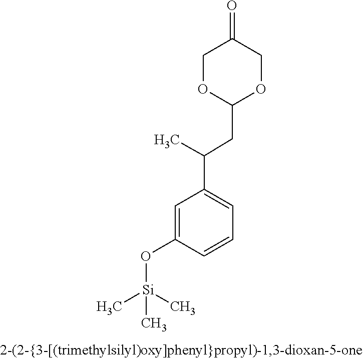 [SMILES: CC(CC1OCC(=O)CO1)c1cccc(O[Si](C)(C)C)c1]

### Example 121

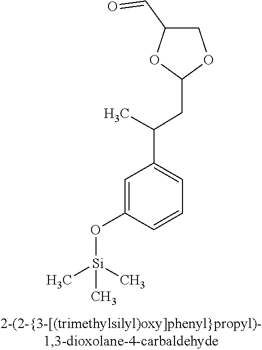 [SMILES: CC(CC1OCC(C=O)O1)c1cccc(O[Si](C)(C)C)c1]

### Example 122

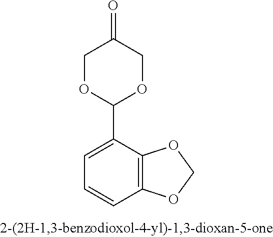 [SMILES: O=C1COC(c2cccc3c2OCO3)OC1]

### Example 123

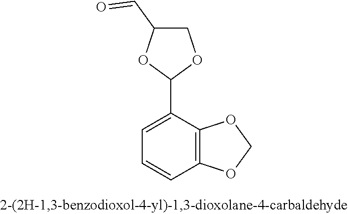 [SMILES: O=CC1COC(c2cccc3c2OCO3)O1]

### Example 124

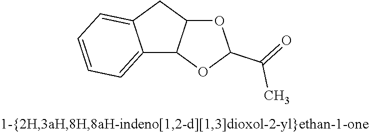

### Example 125

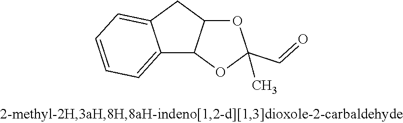

### Example 126

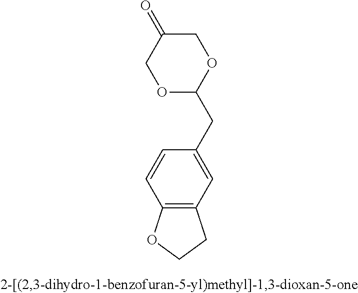 [SMILES: O=C1COC(Cc2ccc3c(c2)CCO3)OC1]

### Example 127

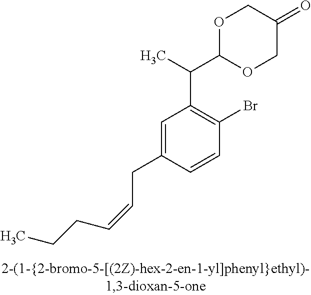 [SMILES: CCC/C=C\Cc1ccc(Br)c(C(C)C2OCC(=O)CO2)c1]

### Example 128

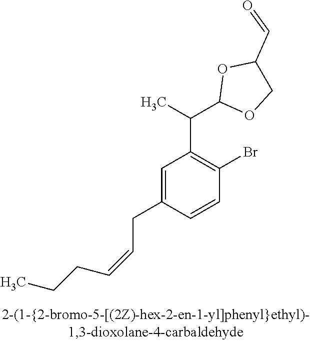 [SMILES: CCC/C=C\Cc1ccc(Br)c(C(C)C2OCC(C=O)O2)c1]

### Example 129

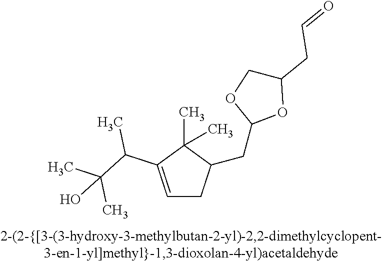 [SMILES: CC(C1=CCC(CC2OCC(CC=O)O2)C1(C)C)C(C)(C)O]

### Example 130

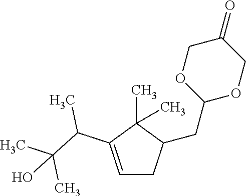 [SMILES: CC(C1=CCC(CC2OCC(=O)CO2)C1(C)C)C(C)(C)O]

### 2-{[3-(3-hydroxy-3-methylbutan-2-yl)-2,2-dimethylcyclopent-3-en-1-yl]methyl}-1,3-dioxan-5-one

In the case of the chemical synthesis reactions described below it is understood that the invention may be synthesized via other reactions. The reactions given herein are provided as examples and should not limit the scope of the invention.

The embodiments detailed below are provided as examples and should not limit the scope of the invention.

### Example 131

The following example is intended to be representative of an embodiment of the present invention. A molecule of the formula:

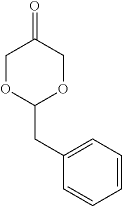 [SMILES: O=C1COC(Cc2ccccc2)OC1]

### 2-benzyl-1,3-dioxan-5-one

In one embodiment, the compound above can be produced via an oxidation of 2-benzyl-1,3-dioxan-5-ol.

2-benzyl-1,3-dioxan-5-ol itself can be produced via a cyclic acetal formation between glycerol and the aldehyde phenylacetaldehyde. In this document it is understood that the term acetal is used as a general term to designate both acetals and ketals.
One of the products from the above reaction is the cyclic acetal, alcohol-containing compound: 2-benzyl-1,3-dioxan-5-ol.
This compound is then oxidized to form the embodiment 2-benzyl-1,3-dioxan-5-one.

The reaction proceeds as in the scheme below.

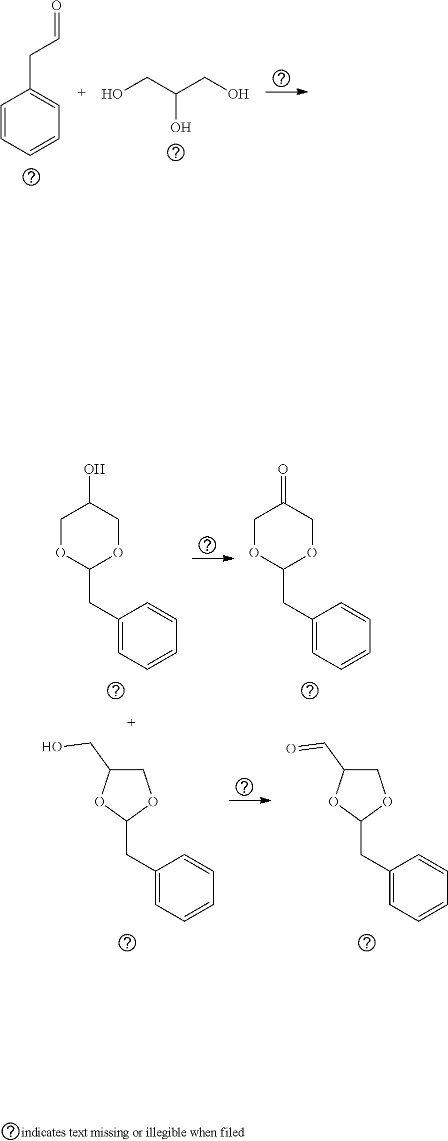 [SMILES: O=CCc1ccccc1 ; OCC(O)CO ; OC1COC(Cc2ccccc2)OC1 ; O=C1COC(Cc2ccccc2)OC1 ; OCC1COC(Cc2ccccc2)O1 ; O=CC1COC(Cc2ccccc2)O1]

2-benzyl-1,3-dioxan-5-one has a very pleasant, characteristic rose-floral aspect. It has been described by some or our reviewers as identical to the smell of a petal of a rose flower with a highly realistic aspect. Others have said it perfectly captures the scent of fresh spring air as mimosa flowers begin to bloom. It is surprisingly much stronger than its starting material and additionally has a much more full and natural-seeming aspect.

The compound is very powerful and diffusive which can offer the advantage of a lower use percentage and thus a lower cost-in-use in formulations. In addition to its use in formulations it can potentially be used as a stand-alone note or single-molecule fragrance.

In other compositions it may provide a base for or aspects of spring air, honeysuckle and other fresh, floral-sweet notes.

In one embodiment, the acetal formation reaction proceeds via toluene reflux. glycerol and phenylacetaldehyde are added in a molar ratio of 2:1. p-toluenesulfonic acid is added at a molar ratio of 0.2 mole as a catalyst. The components are dissolved in toluene or a suitable solvent and the mixture is heated to approximately 110 C. using a Dean-Stark apparatus to remove water resulting from the reaction. The reaction proceeds from 4 hours to overnight. The reaction mixture is then purified and separated to isolate 2-benzyl-1,3-dioxan-5-ol.

The reaction mixture may contain an alternative glyceryl acetal isomer: (2-benzyl-1,3-dioxolan-4-yl)methanol. This compound is also a useful product in its own right and is further detailed herein as a starting material for another embodiment of the invention.

In this embodiment, after 2-benzyl-1,3-dioxan-5-ol is isolated it is oxidized using Dess-Martin Periodinane as an oxidizing reagent. The ratio of Dess-Martin Periodinane to 2-benzyl-1,3-dioxan-5-ol used is 1.5:1. The resulting ketone, 2-benzyl-1,3-dioxan-5-one, is then isolated.

Alternatively, in this embodiment, the commercially available product 2-benzyl-1,3-dioxan-ol or the product “Acetal CD” can be used as a starting material. “Acetal CD” is a mixture of 2-benzyl-1,3-dioxan-5-ol and 2-benzyl-1,3-dioxolan-4-yl)methanol.

In one synthesis method “Acetal CD” (5 g, 25.7 mmol) is dissolved in 100 ml dichloromethane then added Dess-Martin periodinane (17 g, 40 mmol). The mixture solution is then placed under reflux conditions for 12 hours, then poured the solution into 200 mL of ice-cold water and swirled for 10 minutes. After white precipitate forms, the clear solution is collected by vacuum filtration. The clear solution is extracted with 2*20 ml dichloromethane. The organic phase is then washed with brine, dried by Na2SO4, and evaporated under reduced pressure. The residue is purified by flash chromatography (Hexane/EtOAc=20:1) to give 2-benzyl-1,3-dioxan-5-one.

### Alternative Embodiments/Example 132

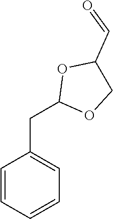 [SMILES: O=CC1COC(Cc2ccccc2)O1]

(“2-benzyl-1,3-dioxolane-4-carbaldehyde”)

The above compound can be prepared as a side product of the synthesis scheme (shown above) for 2-benzyl-1,3-dioxan-5-one. To date it has not been directly isolated but in combination with 2-benzyl-1,3-dioxan-5-one, it appears to act as a modifier and also provides a floral aspect. We are reducing to practice further isolation and evaluation.

It may serve as a useful product in combination 2-benzyl-1,3-dioxan-5-one, which would allow for streamlining the synthesis steps involved in isolating each compound individually. Potentially it may serve as a useful, desirable product in its own right.

### Assorted Additional Examples Structure Images:

The embodiments detailed below are meant to illustrate the range of molecules of which the invention comprises and should not be considered exhaustive.

### Example 133

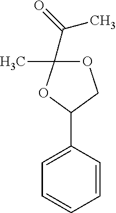

### Example 134

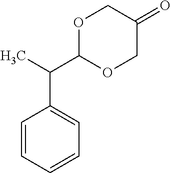

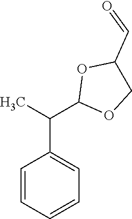

### Example 136

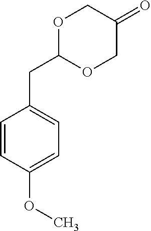 [SMILES: COc1ccc(CC2OCC(=O)CO2)cc1]

### Example 137

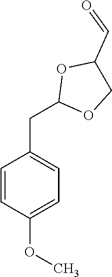 [SMILES: COc1ccc(CC2OCC(C=O)O2)cc1]

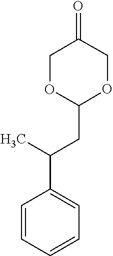 [SMILES: CC(CC1OCC(=O)CO1)c1ccccc1]

### Example 139

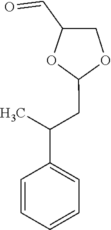 [SMILES: CC(CC1OCC(C=O)O1)c1ccccc1]

### Example 140

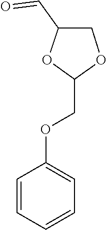 [SMILES: O=CC1COC(COc2ccccc2)O1]

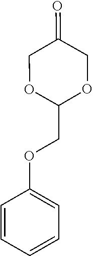 [SMILES: O=C1COC(COc2ccccc2)OC1]

### Example 142

### Example 142

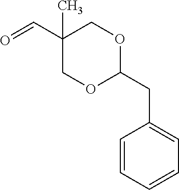 [SMILES: CC1(C=O)COC(Cc2ccccc2)OC1]

### Example 143

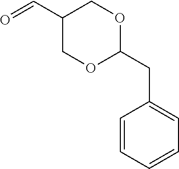 [SMILES: O=CC1COC(Cc2ccccc2)OC1]

### Example 144

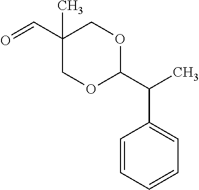 [SMILES: CC(c1ccccc1)C1OCC(C)(C=O)CO1]

### Example 145

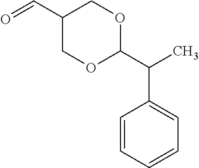 [SMILES: CC(c1ccccc1)C1OCC(C=O)CO1]

### Example 146

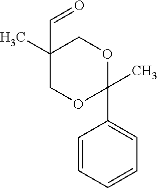

### Example 147

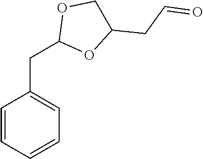 [SMILES: O=CCC1COC(Cc2ccccc2)O1]

### Example 148

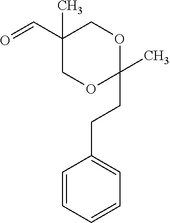 [SMILES: CC1(C=O)COC(C)(CCc2ccccc2)OC1]

### Example 149

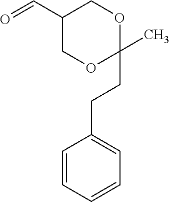 [SMILES: CC1(CCc2ccccc2)OCC(C=O)CO1]

### Example 150

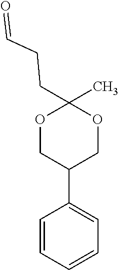 [SMILES: CC1(CCC=O)OCC(c2ccccc2)CO1]

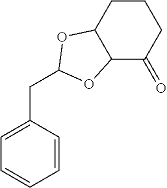 [SMILES: O=C1CCCC2OC(Cc3ccccc3)OC12]

### Example 152

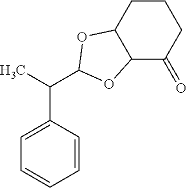 [SMILES: CC(c1ccccc1)C1OC2CCCC(=O)C2O1]

### Example 153

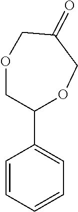 [SMILES: O=C1COCC(c2ccccc2)OC1]

### Example 154

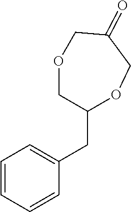 [SMILES: O=C1COCC(Cc2ccccc2)OC1]

### Example 155

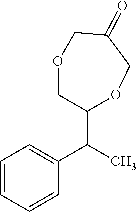 [SMILES: CC(c1ccccc1)C1COCC(=O)CO1]

### Example 156

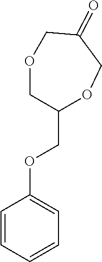 [SMILES: O=C1COCC(COc2ccccc2)OC1]

### Example 157

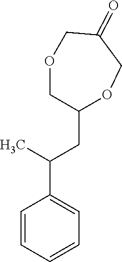 [SMILES: CC(CC1COCC(=O)CO1)c1ccccc1]

### Example 158

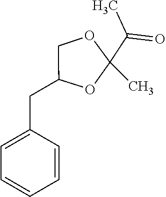 [SMILES: CC(=O)C1(C)OCC(Cc2ccccc2)O1]

### Example 159

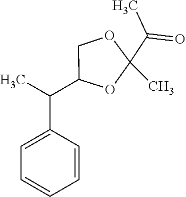 [SMILES: CC(=O)C1(C)OCC(C(C)c2ccccc2)O1]

### Example 160

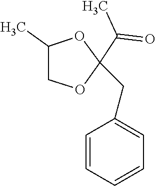 [SMILES: CC(=O)C1(Cc2ccccc2)OCC(C)O1]

### Example 161

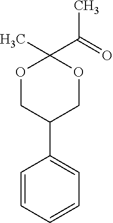 [SMILES: CC(=O)C1(C)OCC(c2ccccc2)CO1]

In an embodiment, the present invention relates to a compound of formula I:

R—(R¹)ₙ—R²—R³ formula I
wherein
R comprises:
   a phenyl group, a C₃₋₆cycloaklyl group, C₃₋₆cycloalkenyl, cyclodienalkyl, adamantyl, naphthalenyl, dihydronaphthalenyl, tetrahydronaphthalenyl, benzodioxolyl, benzofuran, bicyclo[2.2.2]alkyl, bicyclo[3.1.1]alkyl, bicyclo[2.2.2]alkenyl, or bicyclo[3.1.1]alkenyl; wherein R comprises one or more of the following substitutions alone or in combination:
a methyl group,
a two to six carbon alkyl or alkylenyl chain that may be linear, or branched, which is optionally substituted with alkyl groups, a hydroxyl group, or a ketone group,
an oxygen bonded directly to R on one side and bonded to a one- to six-carbon chain that may be linear or branched,
trimethylsilyl,
trimethylsiloxy,
tri-isopropylsiloxy,
a halogen selected from the group consisting of fluorine, chlorine, bromine, and iodine, hydroxyl,
an ester functionality, wherein the ester functionality is either bonded directly to R or contained within a branched or unbranched carbon chain of seven or fewer carbons,
R¹ is a linker moiety selected from the group consisting of a methylene, —O—CH₂—, an ethylene, a propylene, and a butylene group that is optionally substituted with one or more substituents selected from the group consisting of methyl, ethyl, propyl, isopropyl, butyl, isobutyl and t-butyl, and wherein R¹ may optionally contain a C═C double bond,
n is 0 or 1, and when n is 0, R and R² can form a polycyclic structure or a spiro structure,
R² comprises a dioxolane, dioxane or dioxepane group,
R³ comprises a carbonyl moiety that is part of a C₀₋₃ketone or a C₁₋₃aldehyde functionality.

In a variation, the halogen is bromine. In a variation, R is phenyl. In a variation, R² is a dioxolane or a dioxane. In a variation, R is phenyl, naphthalenyl, dihydronaphthalenyl, tetrahydronaphthalenyl. In a variation, R is substituted with a methyl group or a two to six carbon alkyl or alkylenyl chain that may be linear, or branched, which is optionally substituted with alkyl groups, a hydroxyl group, or a ketone group. In a variation, R² is a dioxepane. In a variation, m is 0 and R comprises a spiro or bicyclic moiety. In a variation, m is 1 and the linker is methylene or ethylene, optionally substituted by a methyl group. In a variation, R is substituted by a alkylenyl group that is in a Z configuration.

In an embodiment, the present invention relates to a compound of formula II.

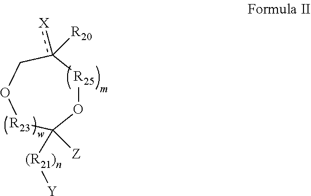

wherein X is selected from the group consisting of O and —(CH₂)ᵥCHO, wherein v is 0, 1, or 2; and when X is 0, R₂₀ is not present;
R₂₀ is selected from the group consisting of H and C₁₋₃alkyl, or R₂₀ is not present; m is 0, 1, or 2, and n is 0, 1, or 2;
R₂₁ is selected from the group consisting of —CH₂—, —R₂₂—, —OR₂₂—, —CH₂R₂₂—, and —R₂₂CH₂— wherein R₂₂ is C₁₋₆alkyl, or C₁₋₃alkylene optionally substituted with a C₁₋₃alkyl;
Y is selected from the group consisting of phenyl, C₃₋₆cycloalkyl, C₃₋₆cycloalkenyl, cyclodienalkyl, adamantyl, naphthalenyl, dihydronaphthalenyl, tetrahydronaphthalenyl, benzodioxolyl, bicyclo[2.2.2]alkyl, bicyclo[3.1.1]alkyl, bicyclo[2.2.2]alkenyl, bicyclo[3.1.1]alkenyl; or when n=0 and Z is not present, Y is a spiro-linked C₃₋₆cycloalkyl, C₃₋₆cycloalkenyl, or cyclodienalkyl; and Z is hydrogen, C₁₋₃alkyl, or not present; wherein Y is optionally substituted with one or more substituents independently selected from the group consisting of hydrogen, hydroxyl, —C(O)OC₁₋₃alkyl, —C₁₋₆alkyl, —C₁₋₆alkenyl, —O—C₁₋₆ alkyl, halogen, or —O—Si(CH₃)₃ wherein the —C₁₋₆alkyl and —C₁₋₆alkenyl as optional substituents off of Y are optionally substituted with hydroxyl, and R₂₃ is (—CH₂—)_{w} wherein w is 0 or 1, or R₂₁, R₂₃, and Y together form a ring structure, R₂₅ is —CH₂— or —CHR₂₆— where R₂₆ is C₁₋₃alkyl.

In a variation, X is O and R₂₀ is not present. In a variation, w is 0 and m is 0 or 1. In a variation, Y is phenyl. In a variation, R₂₁, R₂₃, and Y together form a bicyclic ring structure. In a variation, the compound is selected from the group consisting of:
2-{2-[3-(propan-2-yl)phenyl]propyl}-1,3-dioxan-5-one,
2-{2-[3-(propan-2-yl)phenyl]propyl}-1,3-dioxolane-4-carbaldehyde,
2-[(2-bromophenyl)methyl]-1,3-dioxan-5-one,
2-[(2,2,3-trimethylcyclopent-3-en-1-yl)methyl]-1,3-dioxane-5-carbaldehyde,
2-[(2-bromophenyl)methyl]-1,3-dioxolane-4-carbaldehyde,
2-[(2,4,6-tribromophenyl)methyl]-1,3-dioxan-5-one,
2-[(2,4,6-tribromophenyl)methyl]-1,3-dioxolane-4-carbaldehyde,
2-[(4-bromophenyl)methyl]-1,3-dioxan-5-one,
2-[(4-bromophenyl)methyl]-1,3-dioxolane-4-carbaldehyde,
2-[(4-tert-butylphenyl)methyl]-1,3-dioxan-5-one,
2-[(4-tert-butylphenyl)methyl]-1,3-dioxolane-4-carbaldehyde,
2-(2,2,3,3-tetramethylcyclopropyl)-1,3-dioxan-5-one,
2-[(2,2,3-trimethylcyclopent-3-en-1-yl)methyl]-1,3-dioxan-5-one,
5-methyl-2-[(2,2,3-trimethylcyclopent-3-en-1-yl)methyl]-1,3-dioxane-5-carbaldehyde,
2-[(2,2,3-trimethylcyclopent-3-en-1-yl)methyl]-1,3-dioxolane-4-carbaldehyde,
8,8,10,10-tetramethyl-1,5-dioxaspiro[5.5]undecane-3-carbaldehyde,
3,8,8,10,10-pentamethyl-1,5-dioxaspiro[5.5]undecane-3-carbaldehyde,
8,8,10,10-tetramethyl-1,5-dioxaspiro[5.5]undecan-3-one,
7,7,9,9-tetramethyl-1,4-dioxaspiro[4.5]decane-2-carbaldehyde,
2-(2,4-dimethylcyclohex-3-en-1-yl)-1,3-dioxan-5-one,
2-(2,4-dimethylcyclohex-3-en-1-yl)-1,3-dioxolane-4-carbaldehyde,
2-(2,4-dimethylcyclohex-3-en-1-yl)-1,3-dioxane-5-carbaldehyde,
2-(2,4-dimethylcyclohex-3-en-1-yl)-5-methyl-1,3-dioxane-5-carbaldehyde,
2-[(2,6,6-trimethylcyclohex-1-en-1-yl)methyl]-1,3-dioxan-5-one,
2-[(2,6,6-trimethylcyclohex-1-en-1-yl)methyl]-1,3-dioxolane-4-carbaldehyde,
2-[(2,6,6-trimethylcyclohex-1-en-1-yl)methyl]-1,3-dioxane-5-carbaldehyde,
5-methyl-2-[(2,6,6-trimethylcyclohex-1-en-1-yl)methyl]-1,3-dioxane-5-carbaldehyde,
2-(adamantan-1-yl)-1,3-dioxan-5-one,
2-(adamantan-1-yl)-1,3-dioxolane-4-carbaldehyde,
2-(adamantan-1-yl)-1,3-dioxane-5-carbaldehyde,
2-(adamantan-1-yl)-5-methyl-1,3-dioxane-5-carbaldehyde,
2-(3,3-dimethylcyclohexyl)-2-methyl-1,3-dioxan-5-one,
2-(3,3-dimethylcyclohexyl)-2-methyl-1,3-dioxolane-4-carbaldehyde,
2-(3,3-dimethylcyclohexyl)-2-methyl-1,3-dioxane-5-carbaldehyde,
2-(3,3-dimethylcyclohexyl)-2,5-dimethyl-1,3-dioxane-5-carbaldehyde,
2-[4-(tert-butoxy)phenyl]-1,3-dioxan-5-one,
2-[4-(tert-butoxy)phenyl]-1,3-dioxolane-4-carbaldehyde,
2-[4-(tert-butoxy)phenyl]-1,3-dioxane-5-carbaldehyde,
2-[4-(tert-butoxy)phenyl]-5-methyl-1,3-dioxane-5-carbaldehyde,
2-{4-[(trimethylsilyl)oxy]phenyl}-1,3-dioxan-5-one,
2-{4-[(trimethylsilyl)oxy]phenyl}-1,3-dioxolane-4-carbaldehyde,
2-{4-[(trimethylsilyl)oxy]phenyl}-1,3-dioxane-5-carbaldehyde,
5-methyl-2-{4-[(trimethylsilyl)oxy]phenyl}-1,3-dioxane-5-carbaldehyde,
2-({4-[(trimethylsilyl)oxy]phenyl}methyl)-1,3-dioxan-5-one,
2-({4-[(trimethylsilyl)oxy]phenyl}methyl)-1,3-dioxolane-4-carbaldehyde,
2-({4-[(trimethylsilyl)oxy]phenyl}methyl)-1,3-dioxane-5-carbaldehyde,
5-methyl-2-({4-[(trimethylsilyl)oxy]phenyl}methyl)-1,3-dioxane-5-carbaldehyde,
2-[(2-methylphenyl)methyl]-1,3-dioxan-5-one,
2-[(2-methylphenyl)methyl]-1,3-dioxolane-4-carbaldehyde,
2-[(2-methylphenyl)methyl]-1,3-dioxane-5-carbaldehyde,
5-methyl-2-[(2-methylphenyl)methyl]-1,3-dioxane-5-carbaldehyde,
2-{1-[4-(2-methylpropyl)phenyl]ethyl}-1,3-dioxan-5-one,
2-{1-[4-(2-methylpropyl)phenyl]ethyl}-1,3-dioxolane-4-carbaldehyde,
2-[1-(4-ethylphenyl)ethyl]-1,3-dioxan-5-one,
2-[1-(4-ethylphenyl)ethyl]-1,3-dioxolane-4-carbaldehyde,
2-[1-(4-ethylphenyl)ethyl]-1,3-dioxane-5-carbaldehyde,
2-[1-(4-ethylphenyl)ethyl]-5-methyl-1,3-dioxane-5-carbaldehyde,
2-(2,6,6-trimethylcyclohexa-1,3-dien-1-yl)-1,3-dioxan-5-one,
2-(2,6,6-trimethylcyclohexa-1,3-dien-1-yl)-1,3-dioxolane-4-carbaldehyde,
2-(2,6,6-trimethylcyclohexa-1,3-dien-1-yl)-1,3-dioxane-5-carbaldehyde,
5-methyl-2-(2,6,6-trimethylcyclohexa-1,3-dien-1-yl)-1,3-dioxane-5-carbaldehyde,
2-[(2,6,6-trimethylcyclohexa-1,3-dien-1-yl)methyl]-1,3-dioxan-5-one,
2-[(2,6,6-trimethylcyclohexa-1,3-dien-1-yl)methyl]-1,3-dioxolane-4-carbaldehyde,
2-[(2,6,6-trimethylcyclohexa-1,3-dien-1-yl)methyl]-1,3-dioxane-5-carbaldehyde,
5-methyl-2-[(2,6,6-trimethylcyclohexa-1,3-dien-1-yl)methyl]-1,3-dioxane-5-carbaldehyde,
2-[2-(2-bromophenyl)propyl]-1,3-dioxan-5-one,
2-[2-(2-bromophenyl)propyl]-1,3-dioxolane-4-carbaldehyde,
2-[1-(2-bromophenyl)ethyl]-1,3-dioxan-5-one,
2-[1-(2-bromophenyl)ethyl]-1,3-dioxolane-4-carbaldehyde,
2-[1-(2-bromophenyl)ethyl]-1,3-dioxane-5-carbaldehyde,
2-[1-(2-bromophenyl)ethyl]-5-methyl-1,3-dioxane-5-carbaldehyde,
2-[6-methyl-8-(propan-2-yl)bicyclo[2.2.2]oct-5-en-2-yl]-1,3-dioxan-5-one,
2-[6-methyl-8-(propan-2-yl)bicyclo[2.2.2]oct-5-en-2-yl]-1,3-dioxolane-4-carbaldehyde,
2-[6-methyl-8-(propan-2-yl)bicyclo[2.2.2]oct-5-en-2-yl]-1,3-dioxane-5-carbaldehyde,
5-methyl-2-[6-methyl-8-(propan-2-yl)bicyclo[2.2.2]oct-5-en-2-yl]-1,3-dioxane-5-carbaldehyde,
2-(2-{6,6-dimethylbicyclo[3.1.1]hept-2-en-2-yl}ethyl)-1,3-dioxan-5-one,
2-(2-{6,6-dimethylbicyclo[3.1.1]hept-2-en-2-yl}ethyl)-1,3-dioxolane-4carbaldehyde,
2-(2-{6,6-dimethylbicyclo[3.1.1]hept-2-en-2-yl}ethyl)-1,3-dioxane-5-carbaldehyde,
2-(2-{6,6-dimethylbicyclo[3.1.1]hept-2-en-2-yl}ethyl)-5-methyl-1,3-dioxane-5-carbaldehyde,
2-(2,4,6-trimethylcyclohex-3-en-1-yl)-1,3-dioxan-5-one,
2-(2,4,6-trimethylcyclohex-3-en-1-yl)-1,3-dioxolane-4-carbaldehyde,
2-(2,4,6-trimethylcyclohex-3-en-1-yl)-1,3-dioxane-5-carbaldehyde,
5-methyl-2-(2,4,6-trimethylcyclohex-3-en-1-yl)-1,3-dioxane-5-carbaldehyde,
2-(2,2,3,3-tetramethylcyclopropyl)-1,3-dioxolane-4-carbaldehyde,
2-(2,2,3,3-tetramethylcyclopropyl)-1,3-dioxolane-4-carbaldehyde,
5-methyl-2-(2,2,3,3-tetramethylcyclopropyl)-1,3-dioxane-5-carbaldehyde,
2-[2-(2,2,3,3-tetramethylcyclopropyl)-1,3-dioxolan-4-yl]acetaldehyde,
ethyl 2-formyl-1,4-dioxaspiro[4.5]decane-7-carboxylate,
ethyl 2-(2-oxoethyl)-1,4-dioxaspiro[4.5]decane-7-carboxylate,
ethyl 3-oxo-1,5-dioxaspiro[5.5]undecane-8-carboxylate,
7-methyl-6-[(2Z)-pent-2-en-1-yl]-1,4-dioxaspiro[4.4]non-6-ene-2-carbaldehyde,
2-{7-methyl-6-[(2Z)-pent-2-en-1-yl]-1,4-dioxaspiro[4.4]non-6-en-2-yl}acetaldehyde,
2-methyl-1-[(2Z)-pent-2-en-1-yl]-6,10-dioxaspiro[4.5]dec-1-en-8-one,
2-(4-hydroxy-3-methoxyphenyl)-1,3-dioxan-5-one,
2-(4-hydroxy-3-methoxyphenyl)-1,3-dioxolane-4-carbaldehyde,
2-(4-hydroxy-3-methoxyphenyl)-1,3-dioxane-5-carbaldehyde,
2-(4-hydroxy-3-methoxyphenyl)-5-methyl-1,3-dioxane-5-carbaldehyde,
2-[2-(4-hydroxy-3-methoxyphenyl)-1,3-dioxolan-4-yl]acetaldehyde,
2-(3-ethoxy-4-hydroxyphenyl)-1,3-dioxan-5-one,
2-(3-ethoxy-4-hydroxyphenyl)-1,3-dioxolane-4-carbaldehyde,
2-(2-bromophenyl)-1,4-dioxepan-6-one,
2-[(2-bromophenyl)methyl]-1,4-dioxepan-6-one,
2-[(2-bromo-5-methylphenyl)methyl]-1,4-dioxepan-6-one,
2-({2-bromo-4-[(1E)-3,3-dimethylbut-1-en-1-yl]phenyl}methyl)-1,4-dioxepan-6-one,
2-{2-bromo-5-[(1E)-prop-1-en-1-yl]phenyl}-1,4-dioxepan-6-one,
2-{2-[(2,2,3-trimethylcyclopent-3-en-1-yl)methyl]-1,3-dioxolan-4-yl}acetaldehyde,
2-[(4,4-dimethyl-3,4-dihydronaphthalen-2-yl)methyl]-1,3-dioxan-5-one,
2-[(4-methyl-3,4-dihydronaphthalen-1-yl)methyl]-1,3-dioxan-5-one,
2-(4,4-dimethyl-3,4-dihydronaphthalen-2-yl)-1,3-dioxan-5-one,
2-[2-(4,4-dimethyl-3,4-dihydronaphthalen-2-yl)ethyl]-1,3-dioxan-5-one,
2-(1,2,3,4-tetrahydronaphthalen-2-yl)-1,3-dioxan-5-one,
2-[(naphthalen-2-yl)methyl]-1,3-dioxan-5-one,
2-(naphthalen-2-yl)-1,3-dioxan-5-one,
2-(4-methyl-1,2-dihydronaphthalen-2-yl)-1,3-dioxan-5-one,
2-(naphthalen-1-yl)-1,3-dioxan-5-one,
2-[2-(naphthalen-1-yl)-1,3-dioxolan-4-yl]acetaldehyde,
2-(3,4-dihydronaphthalen-2-yl)-1,3-dioxan-5-one,
2-(3,4-dihydronaphthalen-2-yl)-1,3-dioxolan-4-one,
2-(3,4-dihydronaphthalen-2-yl)-5,5-dimethyl-1,3-dioxolan-4-one,
2-(2-{3-[(trimethylsilyl)oxy]phenyl}propyl)-1,3-dioxan-5-one,
2-(2-{3-[(trimethylsilyl)oxy]phenyl}propyl)-1,3-dioxolane-4-carbaldehyde,
2-(2H-1,3-benzodioxol-4-yl)-1,3-dioxan-5-one,
2-(2H-1,3-benzodioxol-4-yl)-1,3-dioxolane-4-carbaldehyde,
1-{2H,3aH,8H,8aH-indeno[1,2-d][1,3]dioxol-2-yl}ethan-1-one,
2-methyl-2H,3aH,8H,8aH-indeno[1,2-d][1,3]dioxole-2-carbaldehyde,
2-[(2,3-dihydro-1-benzofuran-5-yl)methyl]-1,3-dioxan-5-one,
2-(1-{2-bromo-5-[(2Z)-hex-2-en-1-yl]phenyl}ethyl)-1,3-dioxan-5-one,
2-(1-{2-bromo-5-[(2Z)-hex-2-en-1-yl]phenyl}ethyl)-1,3-dioxolane-4-carbaldehyde,
2-(2-{[3-(3-hydroxy-3-methylbutan-2-yl)-2,2-dimethylcyclopent-3-en-1-yl]methyl}-1,3-dioxolan-4-yl)acetaldehyde, and
2-{[3-(3-hydroxy-3-methylbutan-2-yl)-2,2-dimethylcyclopent-3-en-1-yl]methyl}-1,3-dioxan-5-one.

In an embodiment, the present invention relates to a composition comprising the compounds as enumerated above, wherein the composition further comprises one or more of phthalates, petroleum, parabens, sodium bicarbonate, magnesium hydroxide, a diluent or an excipient.

In an embodiment, the present invention relates to a consumer product comprising the compound as enumerated above. In a variation, the consumer product is a member selected from the group consisting of perfume, soap, shampoo, conditioner, disinfectants laundry detergents fabric softeners dryer sheets carpet freshener, room freshener, scented candle, toilet paper, trash bag, baby product, cologne, after shave, body wash, shaving cream, body lotion, sunscreen, and make-ups/cosmetics. In a variation, the consumer product is perfume and the consumer product further comprises a carrier, the carrier comprising one or more of an alcohol, an alcohol-based solution, or essential oils.

In a variation, the essential oils are one or more members selected from the group consisting of balsam oil, basil oil, bay leaf oil, birch oil, cinnamon oil, citronella oil, cypress oil, clove oil, eucalyptus oil, frankincense oil, geranium oil, grapefruit oil, jasmine oil, jojoba oil, juniper berry oil, lime oil, mandarin oil, myrrh oil, marjoram oil, nutmeg oil, olive oil, oregano oil, peppermint oil, rosemary oil, rose oil, spearmint oil, sage oil, orange oil, tea tree oil, tangerine oil, thyme oil, and vanilla oil or combinations thereof.

In an embodiment, there are a plurality of other chemicals that can be added to generate compositions similar to perfumes including but not limited to linalool, rose oxide, vanillin, geraniol, phenylethanol, limonene, muscone, cyclopentadecanolide, indole, methyl dihydrojasmonate, methyl jasmonate, hydroxycitronellal, citral, benzyl salicylate, methyl anthranilate, benzyl acetate, Iso E Super or Tetramethyl acetyloctahydronaphthalenes, helional, ambroxide, cis-3-hexenol, decanal, dodecanal, acetophenone, gamma-nonalactone, beta-ionone, para-anisaldehyde, or combinations thereof. In a variation, if a perfume is generated, the aroma chemicals in the composition are on the order of 5-20%, 5-40%, or 15-40% by weight, or alternatively, about 20% by weight with the other chemicals comprising the rest. These other chemicals may include, for example, ethanol or low odor carrier oils.

In an embodiment, the present invention relates to methods of making the compounds of the present invention wherein the method involves a) ascertaining via in silico computer docking studies how certain molecules such as dioxolane, dioxane, and dioxepane containing molecules have affinity and can bind at the active site of certain human vomeronasal receptors such as the VN1R1 receptor, and b) generating the dioxolane, dioxane, and dioxepane containing compound by reacting an aldehyde with a glycerol or an alkyl triol compound. In a variation, the aldehyde is attached directly to a phenyl group or the aldehyde is attached to a phenyl group through a linker that is comprised of an alkylene functionality. In an embodiment, other ring structures can be bonded either directly or indirectly through an alkyl linker to the aldehyde substituent.

In an embodiment, the present invention relates to a method of developing ligands/compounds that fit within the active site of vomeronasal. In a variation, the vomeronasal receptor is the VN1R1 receptor. The method allows one to find ligands/compounds that fit in the active site in such a way that they should provide smell. In an embodiment, in silico docking simulations can be performed to ascertain if the compounds will be odorants. In a variation, the in silico docking simulations can be done in triplicate and predicted affinities can be calculated using the various simulations. In a variation, a predicted binding affinity of −5 kcal/mol (average of three repetitions) or lower (i.e., a bigger negative number) indicates moderately good binding to the human VN1R1 receptor. Thus, in an embodiment, the method comprises doing computer simulations to ascertain a plurality of candidate compounds that should exhibit decent binding characteristics to one or more vomeronasal receptors. After performing in silico docking simulations of these candidate compounds in the receptors and predicting their respective affinities, the compounds with the best affinities can be synthesized using the procedures as disclosed herein or alternate methods. It is expected that both the size of the compounds and the composition of the various parts of the compounds (e.g., the electrostatic characteristics of the compounds) will allow ideal candidates to be found. For example, if a hydroxyl group (for example from a serine residue) is present in the active site, one might expect to attain decent to good binding characteristics with the oxygen in an acetal group in a compound through hydrogen bonding (from the hydroxyl hydrogen and the acetal oxygen) that is correctly positioned. The carbonyl structure on the aldehydes and/or ketones disclosed herein may also provide possible additional binding sites. Similarly, hydrophobic portions on candidate compounds may associate with hydrophobic side chains in the active site (for example from leucine, isoleucine, valine or other amino acids with hydrophobic side chains).

The in silico docking simulations can be utilized as a novel screening tool, to allow for rapid changes in size and compositional make-up of candidate compounds allowing for potential odorous compounds to be found easily, quickly and efficiently. Additionally, using free energy perturbations, ensemble docking, and other related methods of in silico docking allow one to identify the best possible candidates more comprehensively and with greater relevance to the receptor protein in question. In a variation, once one has synthesized the various compounds (using methods as disclosed herein), testing can be done to ascertain the pleasantness or unpleasantness of the various synthesized compounds. It should be understood that as additional data is attained by ascertaining the pleasantness or lack thereof, the in silico docking program can be modified to account for not just binding affinity, but also for the shape and the electrostatic characteristics of compounds that are to be tested. In a variation, AI or Machine Learning may be used in conjunction with the in silico docking program so that as more data is gained, the program can make predictions about compounds that may be best suited to attain a particular characteristic and subsequently generate candidate compounds with the highest predicted binding affinity or activation of a given olfactory receptor.

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

We claim:

1) A compound of formula I:
R—(R¹)ₙ—R²—R³ formula I
wherein
R comprises:
a phenyl group, a C₃₋₆cycloaklyl group, C₃₋₆cycloalkenyl, cyclodienalkyl, adamantyl, naphthalenyl, dihydronaphthalenyl, tetrahydronaphthalenyl, benzodioxolyl, benzofuran, bicyclo[2.2.2]alkyl, bicyclo[3.1.1]alkyl, bicyclo[2.2.2]alkenyl, or bicyclo[3.1.1]alkenyl; wherein R comprises one or more of the following substitutions alone or in combination:
a methyl group,
a two to six carbon alkyl or alkylenyl chain that may be linear, or branched, which is optionally substituted with alkyl groups, a hydroxyl group, or a ketone group,
an oxygen bonded directly to R on one side and bonded to a one- to six-carbon chain that may be linear or branched,
trimethylsilyl,
trimethylsiloxy,
tri-isopropylsiloxy,
a halogen selected from the group consisting of fluorine, chlorine, bromine, and iodine,
hydroxyl,
an ester functionality, wherein the ester functionality is either bonded directly to R or contained within a branched or unbranched carbon chain of seven or fewer carbons,
R¹ is a linker moiety selected from the group consisting of a methylene, —O—CH₂—, an ethylene, a propylene, and a butylene group that is optionally substituted with one or more substituents selected from the group consisting of methyl, ethyl, propyl, isopropyl, butyl, isobutyl and t-butyl, and wherein R¹ may optionally contain a C═C double bond,
n is 0 or 1, and when n is 0, R and R² can form a polycyclic structure or a spiro structure,
R² comprises a dioxolane, dioxane or dioxepane group,
R³ comprises a carbonyl moiety that is part of a C₀₋₃ketone or a C₁₋₃aldehyde functionality.

2) The compound of claim 1, wherein the halogen is bromine.

3) The compound of claim 1, wherein R is phenyl.

4) The compound of claim 1, wherein R² is a dioxolane or a dioxane.

5) The compound of claim 1, wherein R is phenyl, naphthalenyl, dihydronaphthalenyl, tetrahydronaphthalenyl.
The compound of claim 1, wherein R is substituted with a methyl group or a two to six carbon alkyl or alkylenyl chain that may be linear, or branched, which is optionally substituted with alkyl groups, a hydroxyl group, or a ketone group.

6) The compound of claim 1, wherein R2 is a dioxepane.

7) The compound of claim 1, wherein m is 0 and R comprises a spiro or bicyclic moiety.

8) The compound of claim 1, wherein m is 1 and the linker is methylene or ethylene, optionally substituted by a methyl group.

9) The compound of claim 1, wherein R is substituted by a alkylenyl group that is in a Z configuration.

10) A compound of formula II: 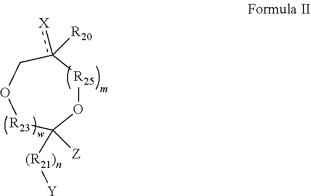
wherein X is selected from the group consisting of O and —(CH₂)ᵥCHO, wherein v is 0, 1, or 2; and when X is 0, R₂₀ is not present;
R₂₀ is selected from the group consisting of H and C₁₋₃alkyl, or R₂₀ is not present; m is 0, 1, or 2, and n is 0, 1, or 2;
R₂₁ is selected from the group consisting of —CH₂—, —R₂₂—, —OR₂₂—, —CH₂R₂₂—, and —R₂₂CH₂— wherein R₂₂ is C₁₋₆alkyl, or C₁₋₃alkylene optionally substituted with a C₁₋₃alkyl;
Y is selected from the group consisting of phenyl, C₃₋₆cycloalkyl, C₃₋₆cycloalkenyl, cyclodienalkyl, adamantyl, naphthalenyl, dihydronaphthalenyl, tetrahydronaphthalenyl, benzodioxolyl, bicyclo[2.2.2]alkyl, bicyclo[3.1.1]alkyl, bicyclo[2.2.2]alkenyl, bicyclo[3.1.1]alkenyl; or when n=0 and Z is not present, Y is a spiro-linked C₃₋₆cycloalkyl, C₃₋₆cycloalkenyl, or cyclodienalkyl; and Z is hydrogen, C₁₋₃alkyl, or not present; wherein Y is optionally substituted with one or more substituents independently selected from the group consisting of hydrogen, hydroxyl, —C(O)OC₁₋₃alkyl, —C₁₋₆alkyl, —C₁₋₆alkenyl, —O—C₁₋₆alkyl, halogen, or —O—Si(CH₃)₃ wherein the —C₁₋₆alkyl and —C₁₋₆alkenyl as optional substituents off of Y are optionally substituted with hydroxyl, and R₂₃ is (—CH₂—)_{w} wherein w is 0 or 1, or R₂₁, R₂₃, and Y together form a ring structure, R₂₅ is —CH₂— or —CHR₂₆— where R₂₆ is C₁₋₃alkyl.

11) The compound of claim 10, wherein X is O and R₂₀ is not present.

12) The compound of claim 10, wherein w is 0 and m is 0 or 1.

13) The compound of claim 10, wherein Y is phenyl.

14) The compound of claim 10, wherein R₂₁, R₂₃, and Y together form a bicyclic ring structure.

15) The compound of claim 10, wherein the compound is selected from the group consisting of:
2-{2-[3-(propan-2-yl)phenyl]propyl}-1,3-dioxan-5-one,
2-{2-[3-(propan-2-yl)phenyl]propyl}-1,3-dioxolane-4-carbaldehyde,
2-[(2-bromophenyl)methyl]-1,3-dioxan-5-one,
2-[(2,2,3-trimethylcyclopent-3-en-1-yl)methyl]-1,3-dioxane-5-carbaldehyde,
2-[(2-bromophenyl)methyl]-1,3-dioxolane-4-carbaldehyde,
2-[(2,4,6-tribromophenyl)methyl]-1,3-dioxan-5-one,
2-[(2,4,6-tribromophenyl)methyl]-1,3-dioxolane-4-carbaldehyde,
2-[(4-bromophenyl)methyl]-1,3-dioxan-5-one,
2-[(4-bromophenyl)methyl]-1,3-dioxolane-4-carbaldehyde,
2-[(4-tert-butylphenyl)methyl]-1,3-dioxan-5-one,
2-[(4-tert-butylphenyl)methyl]-1,3-dioxolane-4-carbaldehyde,
2-(2,2,3,3-tetramethylcyclopropyl)-1,3-dioxan-5-one,
2-[(2,2,3-trimethylcyclopent-3-en-1-yl)methyl]-1,3-dioxan-5-one,
5-methyl-2-[(2,2,3-trimethylcyclopent-3-en-1-yl)methyl]-1,3-dioxane-5-carbaldehyde,
2-[(2,2,3-trimethylcyclopent-3-en-1-yl)methyl]-1,3-dioxolane-4-carbaldehyde,
8,8,10,10-tetramethyl-1,5-dioxaspiro[5.5]undecane-3-carbaldehyde,
3,8,8,10,10-pentamethyl-1,5-dioxaspiro[5.5]undecane-3-carbaldehyde,
8,8,10,10-tetramethyl-1,5-dioxaspiro[5.5]undecan-3-one,
7,7,9,9-tetramethyl-1,4-dioxaspiro[4.5]decane-2-carbaldehyde,
2-(2,4-dimethylcyclohex-3-en-1-yl)-1,3-dioxan-5-one,
2-(2,4-dimethylcyclohex-3-en-1-yl)-1,3-dioxolane-4-carbaldehyde,
2-(2,4-dimethylcyclohex-3-en-1-yl)-1,3-dioxane-5-carbaldehyde,
2-(2,4-dimethylcyclohex-3-en-1-yl)-5-methyl-1,3-dioxane-5-carbaldehyde,
2-[(2,6,6-trimethylcyclohex-1-en-1-yl)methyl]-1,3-dioxan-5-one,
2-[(2,6,6-trimethylcyclohex-1-en-1-yl)methyl]-1,3-dioxolane-4-carbaldehyde,
2-[(2,6,6-trimethylcyclohex-1-en-1-yl)methyl]-1,3-dioxane-5-carbaldehyde,
5-methyl-2-[(2,6,6-trimethylcyclohex-1-en-1-yl)methyl]-1,3-dioxane-5-carbaldehyde,
2-(adamantan-1-yl)-1,3-dioxan-5-one,
2-(adamantan-1-yl)-1,3-dioxolane-4-carbaldehyde,
2-(adamantan-1-yl)-1,3-dioxane-5-carbaldehyde,
2-(adamantan-1-yl)-5-methyl-1,3-dioxane-5-carbaldehyde,
2-(3,3-dimethylcyclohexyl)-2-methyl-1,3-dioxan-5-one,
2-(3,3-dimethylcyclohexyl)-2-methyl-1,3-dioxolane-4-carbaldehyde,
2-(3,3-dimethylcyclohexyl)-2-methyl-1,3-dioxane-5-carbaldehyde,
2-(3,3-dimethylcyclohexyl)-2,5-dimethyl-1,3-dioxane-5-carbaldehyde,
2-[4-(tert-butoxy)phenyl]-1,3-dioxan-5-one,
2-[4-(tert-butoxy)phenyl]-1,3-dioxolane-4-carbaldehyde,
2-[4-(tert-butoxy)phenyl]-1,3-dioxane-5-carbaldehyde,
2-[4-(tert-butoxy)phenyl]-5-methyl-1,3-dioxane-5-carbaldehyde,
2-{4-[(trimethylsilyl)oxy]phenyl}-1,3-dioxan-5-one,
2-{4-[(trimethylsilyl)oxy]phenyl}-1,3-dioxolane-4-carbaldehyde,
2-{4-[(trimethylsilyl)oxy]phenyl}-1,3-dioxane-5-carbaldehyde,
5-methyl-2-{4-[(trimethylsilyl)oxy]phenyl}-1,3-dioxane-5-carbaldehyde,
2-({4-[(trimethylsilyl)oxy]phenyl}methyl)-1,3-dioxan-5-one,
2-({4-[(trimethylsilyl)oxy]phenyl}methyl)-1,3-dioxolane-4-carbaldehyde,
2-({4-[(trimethylsilyl)oxy]phenyl}methyl)-1,3-dioxane-5-carbaldehyde,
5-methyl-2-({4-[(trimethylsilyl)oxy]phenyl}methyl)-1,3-dioxane-5-carbaldehyde,
2-[(2-methylphenyl)methyl]-1,3-dioxan-5-one,
2-[(2-methylphenyl)methyl]-1,3-dioxolane-4-carbaldehyde,
2-[(2-methylphenyl)methyl]-1,3-dioxane-5-carbaldehyde,
5-methyl-2-[(2-methylphenyl)methyl]-1,3-dioxane-5-carbaldehyde,
2-{1-[4-(2-methylpropyl)phenyl]ethyl}-1,3-dioxan-5-one,
2-{1-[4-(2-methylpropyl)phenyl]ethyl}-1,3-dioxolane-4-carbaldehyde,
2-[1-(4-ethylphenyl)ethyl]-1,3-dioxan-5-one,
2-[1-(4-ethylphenyl)ethyl]-1,3-dioxolane-4-carbaldehyde,
2-[1-(4-ethylphenyl)ethyl]-1,3-dioxane-5-carbaldehyde,
2-[1-(4-ethylphenyl)ethyl]-5-methyl-1,3-dioxane-5-carbaldehyde,
2-(2,6,6-trimethylcyclohexa-1,3-dien-1-yl)-1,3-dioxan-5-one,
2-(2,6,6-trimethylcyclohexa-1,3-dien-1-yl)-1,3-dioxolane-4-carbaldehyde,
2-(2,6,6-trimethylcyclohexa-1,3-dien-1-yl)-1,3-dioxane-5-carbaldehyde,
5-methyl-2-(2,6,6-trimethylcyclohexa-1,3-dien-1-yl)-1,3-dioxane-5-carbaldehyde,
2-[(2,6,6-trimethylcyclohexa-1,3-dien-1-yl)methyl]-1,3-dioxan-5-one,
2-[(2,6,6-trimethylcyclohexa-1,3-dien-1-yl)methyl]-1,3-dioxolane-4-carbaldehyde,
2-[(2,6,6-trimethylcyclohexa-1,3-dien-1-yl)methyl]-1,3-dioxane-5-carbaldehyde,
5-methyl-2-[(2,6,6-trimethylcyclohexa-1,3-dien-1-yl)methyl]-1,3-dioxane-5-carbaldehyde,
2-[2-(2-bromophenyl)propyl]-1,3-dioxan-5-one,
2-[2-(2-bromophenyl)propyl]-1,3-dioxolane-4-carbaldehyde,
2-[1-(2-bromophenyl)ethyl]-1,3-dioxan-5-one,
2-[1-(2-bromophenyl)ethyl]-1,3-dioxolane-4-carbaldehyde,
2-[1-(2-bromophenyl)ethyl]-1,3-dioxane-5-carbaldehyde,
2-[1-(2-bromophenyl)ethyl]-5-methyl-1,3-dioxane-5-carbaldehyde,
2-[6-methyl-8-(propan-2-yl)bicyclo[2.2.2]oct-5-en-2-yl]-1,3-dioxan-5-one,
2-[6-methyl-8-(propan-2-yl)bicyclo[2.2.2]oct-5-en-2-yl]-1,3-dioxolane-4-carbaldehyde,
2-[6-methyl-8-(propan-2-yl)bicyclo[2.2.2]oct-5-en-2-yl]-1,3-dioxane-5-carbaldehyde,
5-methyl-2-[6-methyl-8-(propan-2-yl)bicyclo[2.2.2]oct-5-en-2-yl]-1,3-dioxane-5-carbaldehyde,
2-(2-{6,6-dimethylbicyclo[3.1.1]hept-2-en-2-yl}ethyl)-1,3-dioxan-5-one,
2-(2-{6,6-dimethylbicyclo[3.1.1]hept-2-en-2-yl}ethyl)-1,3-dioxolane-4carbaldehyde,
2-(2-{6,6-dimethylbicyclo[3.1.1]hept-2-en-2-yl}ethyl)-1,3-dioxane-5-carbaldehyde,
2-(2-{6,6-dimethylbicyclo[3.1.1]hept-2-en-2-yl}ethyl)-5-methyl-1,3-dioxane-5-carbaldehyde,
2-(2,4,6-trimethylcyclohex-3-en-1-yl)-1,3-dioxan-5-one,
2-(2,4,6-trimethylcyclohex-3-en-1-yl)-1,3-dioxolane-4-carbaldehyde,
2-(2,4,6-trimethylcyclohex-3-en-1-yl)-1,3-dioxane-5-carbaldehyde,
5-methyl-2-(2,4,6-trimethylcyclohex-3-en-1-yl)-1,3-dioxane-5-carbaldehyde,
2-(2,2,3,3-tetramethylcyclopropyl)-1,3-dioxolane-4-carbaldehyde,
2-(2,2,3,3-tetramethylcyclopropyl)-1,3-dioxolane-4-carbaldehyde,
5-methyl-2-(2,2,3,3-tetramethylcyclopropyl)-1,3-dioxane-5-carbaldehyde,
2-[2-(2,2,3,3-tetramethylcyclopropyl)-1,3-dioxolan-4-yl]acetaldehyde,
ethyl 2-formyl-1,4-dioxaspiro[4.5]decane-7-carboxylate,
ethyl 2-(2-oxoethyl)-1,4-dioxaspiro[4.5]decane-7-carboxylate,
ethyl 3-oxo-1,5-dioxaspiro[5.5]undecane-8-carboxylate,
7-methyl-6-[(2Z)-pent-2-en-1-yl]-1,4-dioxaspiro[4.4]non-6-ene-2-carbaldehyde,
2-{7-methyl-6-[(2Z)-pent-2-en-1-yl]-1,4-dioxaspiro[4.4]non-6-en-2-yl}acetaldehyde,
2-methyl-1-[(2Z)-pent-2-en-1-yl]-6,10-dioxaspiro[4.5]dec-1-en-8-one,
2-(4-hydroxy-3-methoxyphenyl)-1,3-dioxan-5-one,
2-(4-hydroxy-3-methoxyphenyl)-1,3-dioxolane-4-carbaldehyde,
2-(4-hydroxy-3-methoxyphenyl)-1,3-dioxane-5-carbaldehyde,
2-(4-hydroxy-3-methoxyphenyl)-5-methyl-1,3-dioxane-5-carbaldehyde,
2-[2-(4-hydroxy-3-methoxyphenyl)-1,3-dioxolan-4-yl]acetaldehyde,
2-(3-ethoxy-4-hydroxyphenyl)-1,3-dioxan-5-one,
2-(3-ethoxy-4-hydroxyphenyl)-1,3-dioxolane-4-carbaldehyde,
2-(2-bromophenyl)-1,4-dioxepan-6-one,
2-[(2-bromophenyl)methyl]-1,4-dioxepan-6-one,
2-[(2-bromo-5-methylphenyl)methyl]-1,4-dioxepan-6-one,
2-({2-bromo-4-[(1E)-3,3-dimethylbut-1-en-1-yl]phenyl}methyl)-1,4-dioxepan-6-one,
2-{2-bromo-5-[(1E)-prop-1-en-1-yl]phenyl}-1,4-dioxepan-6-one,
2-{2-[(2,2,3-trimethylcyclopent-3-en-1-yl)methyl]-1,3-dioxolan-4-yl}acetaldehyde,
2-[(4,4-dimethyl-3,4-dihydronaphthalen-2-yl)methyl]-1,3-dioxan-5-one,
2-[(4-methyl-3,4-dihydronaphthalen-1-yl)methyl]-1,3-dioxan-5-one,
2-(4,4-dimethyl-3,4-dihydronaphthalen-2-yl)-1,3-dioxan-5-one,
2-[2-(4,4-dimethyl-3,4-dihydronaphthalen-2-yl)ethyl]-1,3-dioxan-5-one,
2-(1,2,3,4-tetrahydronaphthalen-2-yl)-1,3-dioxan-5-one,
2-[(naphthalen-2-yl)methyl]-1,3-dioxan-5-one,
2-(naphthalen-2-yl)-1,3-dioxan-5-one,
2-(4-methyl-1,2-dihydronaphthalen-2-yl)-1,3-dioxan-5-one,
2-(naphthalen-1-yl)-1,3-dioxan-5-one,
2-[2-(naphthalen-1-yl)-1,3-dioxolan-4-yl]acetaldehyde,
2-(3,4-dihydronaphthalen-2-yl)-1,3-dioxan-5-one,
2-(3,4-dihydronaphthalen-2-yl)-1,3-dioxolan-4-one,
2-(3,4-dihydronaphthalen-2-yl)-5,5-dimethyl-1,3-dioxolan-4-one,
2-(2-{3-[(trimethylsilyl)oxy]phenyl}propyl)-1,3-dioxan-5-one,
2-(2-{3-[(trimethylsilyl)oxy]phenyl}propyl)-1,3-dioxolane-4-carbaldehyde,
2-(2H-1,3-benzodioxol-4-yl)-1,3-dioxan-5-one,
2-(2H-1,3-benzodioxol-4-yl)-1,3-dioxolane-4-carbaldehyde,
1-{2H,3aH,8H,8aH-indeno[1,2-d][1,3]dioxol-2-yl}ethan-1-one,
2-methyl-2H,3aH,8H,8aH-indeno[1,2-d][1,3]dioxole-2-carbaldehyde,
2-[(2,3-dihydro-1-benzofuran-5-yl)methyl]-1,3-dioxan-5-one,
2-(1-{2-bromo-5-[(2Z)-hex-2-en-1-yl]phenyl}ethyl)-1,3-dioxan-5-one,
2-(1-{2-bromo-5-[(2Z)-hex-2-en-1-yl]phenyl}ethyl)-1,3-dioxolane-4-carbaldehyde,
2-(2-{[3-(3-hydroxy-3-methylbutan-2-yl)-2,2-dimethylcyclopent-3-en-1-yl]methyl}-1,3-dioxolan-4-yl)acetaldehyde, and
2-{[3-(3-hydroxy-3-methylbutan-2-yl)-2,2-dimethylcyclopent-3-en-1-yl]methyl}-1,3-dioxan-5-one.

16) A composition comprising the compound of claim 10, wherein the composition further comprises one or more of phthalates, petroleum, parabens, sodium bicarbonate, magnesium hydroxide, a diluent or an excipient.

17) A consumer product comprising the compound of claim 10.

18) The consumer product of claim 16, wherein the consumer product is a member selected from the group consisting of perfume, soap, shampoo, conditioner, disinfectants laundry detergents fabric softeners dryer sheets carpet freshener, room freshener, scented candle, toilet paper, trash bag, baby product, cologne, after shave, body wash, shaving cream, body lotion, sunscreen, and make-ups/cosmetics.

19) The consumer product of claim 17, wherein the consumer product is perfume and the consumer product further comprises a carrier, the carrier comprising one or more of an alcohol, an alcohol based solution, or essential oils.

20) The consumer product of claim 19, wherein the essential oils are one or more members selected from the group consisting of balsam oil, basil oil, bay leaf oil, birch oil, cinnamon oil, citronella oil, cypress oil, clove oil, eucalyptus oil, frankincense oil, geranium oil, grapefruit oil, jasmine oil, jojoba oil, juniper berry oil, lime oil, mandarin oil, myrrh oil, marjoram oil, nutmeg oil, olive oil, oregano oil, peppermint oil, rosemary oil, rose oil, spearmint oil, sage oil, orange oil, tea tree oil, tangerine oil, thyme oil, and vanilla oil or combinations thereof.

21) A method for improving or modifying the olfactory properties of a starting molecule that contains an aldehyde or ketone functional group by converting said aldehyde or ketone to a dioxolane, dioxane or dioxepane structure wherein said dioxolane, dioxane or dioxepane structure also has a carbonyl moiety associated with the dioxolane, dioxane or dioxepane structure or that is part of a separate ketone or C₁₋₃ aldehyde functionality directly bonded to the dioxolane, dioxane or dioxepane structure.

22) A method of developing one or more odorous compounds via in silico docking, said method comprising using data pertaining to a spatial relationship of atoms at an active site of an olfactory receptor, using computer docking software to place candidate compounds in the active site of the olfactory receptor, and instructing the computer docking software to perform calculations to ascertain binding affinity, electrostatic relationships and/or spatial relationships between the candidate compounds and the olfactory receptor.

23) The method of claim 22, wherein the olfactory receptor is a vomeronasal receptor, selected from the group consisting of VN1R1, VN1R2, VN1R3, and VN1R5.

24) The method of claim 23, further comprising using physical assays in conjunction with in silico docking for a purpose of targeting the vomeronasal receptor in order to develop, modify, screen, or improve the one or more odorous compounds.
